(19) United States
(12) Patent Application Publication
(10) Pub. No.: US 2013/0218045 A1
(43) Pub. Date: Aug. 22, 2013

(54) Method and System for Disease Risk Management

(71) Applicant: Joel Ironstone, Toronto (CA)

(72) Inventor: Joel Ironstone, Toronto (CA)

(21) Appl. No.: 13/591,914

(22) Filed: Aug. 22, 2012

Related U.S. Application Data
(60) Provisional application No. 61/525,887, filed on Aug. 22, 2011.
(60) Provisional application No. 61/561,978, filed on Nov. 21, 2011.
(60) Provisional application No. 61/616,083, filed on Mar. 27, 2012.

(51) Int. Cl. A61B 5/053

(52) U.S. Cl. CPC A61B 5/0537
USPC 600/547

(57) ABSTRACT

This disclosure relates to systems and methods for measuring the composition of a body part of a patient, or for otherwise determining a clinically relevant fact using characteristics of tissue in or proximal to said body part. In an example, this disclosure relates to measuring the electrical impedance of an organ or portion of an organ, such as the female breast, so as to obtain clinically relevant information. In an aspect, the system and method can be used for measuring and utilizing certain information such as breast density data and other risk factors for determination or classification of a woman's likelihood to develop breast cancer. Other aspects quantify or qualify a woman's responsiveness to a drug or hormonal therapy.

### RELATED APPLICATIONS

This application claims the benefit and priority, under US and PCT statutes, of U.S. Provisional Application 61/525,887 entitled “System and Method for Non-Invasive Measurement of Composition of a Body Part Using Electrical Characteristics Thereof”, filed on Aug. 22, 2011; 61/561,978 entitled “System and Method for Monitoring Risk of Disease in a Body Part Using Electrical Characteristics Thereof”, filed on Nov. 21, 2011; and 61/616,083 entitled “Method and System for Disease Risk Assessment”, filed on Mar. 27, 2012, which are all hereby incorporated by reference.

### TECHNICAL FIELD

The present disclosure relates to systems and methods for measuring the composition of a body part of a patient, which may be a human breast, or for otherwise determining a clinically relevant fact, which may be a risk of future cancer in said body part, using non-invasive measurement of characteristics of tissue in or proximal to said body part, which may be a measured electrical impedance of the body part.

### BACKGROUND

Sophisticated clinical techniques have been employed in the effort to detect and reduce the mortality of certain diseases such as cancer. It is known that early detection of many types of cancers can significantly improve a patient's chances for successful treatment and survival. Therefore, patients are sometimes screened for a disease or indicia of the disease, especially if the patients have a history relevant to developing or having the disease. In cancer screening, the screening is intended to detect at an early stage any sign of cancerous or pre-cancerous activity in one or more target organs of the patient.

Breast cancer in women is a major health concern from a social and economic point of view. Breast cancer manifests itself primarily in the female breast tissue, and once firmly established, it can be readily identified by a qualified physician, and in some cases by the patient herself. Various methods are used to screen women for the onset of breast cancer, especially those with known or suspected risk factors. Breast cancer also exists in men, albeit a less substantial public health concern than female breast cancer. Men are typically not routinely screened for breast cancer. In women, presently employed methods for screening for breast cancer are generally too expensive, insensitive, slow, inconvenient, painful, or a combination of the above.

Breast cancer is the most commonly diagnosed cancer in women, and the second most deadly after lung cancer. A Canadian woman has a 1 in 9 lifetime risk of being diagnosed with breast cancer and a 1 in 28 lifetime risk of dying from it. Mammographic screening programs have been proven to significantly reduce breast cancer mortality. Mammographic screening is recommended for women 50 years old and above in Canada and for women 40 and over in the U.S. The U.S. preventative task force has recommended annual screening mammograms only for women 50 years and over, with biannual mammograms in the 40 to 50 year cohort based on the individuals risk tolerance. The change in recommendation is based on the high likelihood of false positives and the relative paucity of cancers in that cohort. It is well established that if higher risk groups can be identified, additional screening resources should be spent on them. For example, in women who are at elevated (approximately 20-25%) lifetime risk of breast cancer, earlier screening and screening with alternative technologies such as MRI is indicated beginning at age 30.

The relative risk to an individual with the breast cancer related genes BRCA1 and BRCA2 to develop the disease is 8-10 fold increased. However, the breast cancer genes are rare, and less than 5% of breast cancers are attributable to either mutation. In comparison, breast density has been shown to account for 26-40% of all breast cancers in younger women. There is a 4-6 fold increase in cancer risk related to greater breast density. Breast density is consistently associated with breast cancer risk, more strongly than most other risk factors for this disease, and increased breast density may account for a substantial fraction of breast cancer. Current models for estimating lifetime risk include family history, personal information, previous breast disease, and hormonal/reproductive factors. Each of these individual factors has a relative risk of 1.5 to 3. Breast density, with a relative risk of up to 6, is the single most predictive factor for breast cancer, and is not included in any of these models.

Unlike other predictive criteria for breast cancer such as age of menarche, hormonal history, and genetics, breast density can be modified. Factors that have been clinically shown to modify breast density include vitamin intake and use of hormone replacement therapy (HRT), cancer therapy drugs, alcohol consumption, and possibly exercise. It has also been shown that when breast density is modified, there is a commensurate change in the individual's breast cancer risk.

Currently, Mammograms are scored using the BI-RADS system for breast density assessment that involves four qualitative categories, which are reader-dependent. The categories are: almost entirely fat (I), scattered fibroglandular densities (II), moderately dense (III), extremely dense (IV). The lack of well defined categories means that inter-reader variability can be significant. The method is additionally limited by the number of categories and the more categories involved in the breast density assessment, the more predictive of the individual's risk. More quantitative measures of assessing breast density from mammograms have been developed involving human and computer aided detection methods. Recently, computer aided design (CAD) and similar software has been approved by the FDA for the estimation of breast density on a digital mammogram. This and other 2-D mammographic methods of assessing breast density are sometimes limited by the fact that what is being measured is a 2-D projection of a 3-D volume which is further obfuscated by variation in parameters such as breast positioning and compression rates.

Other techniques for measuring the relevant quantities are invasive (require penetration of a human body) or ionizing (utilize ionizing radiation) and as such are not ideal for practicing on the bodies of younger patients who are not known to actually have the cancer disease or require regular or frequent application of invasive and/or ionizing methods.

A mammographic-based method of detecting breast density is not usually available to anyone who is not currently undergoing mammographic screening. Dual X-Ray absorptiometry (DXA) systems are currently indicated for whole body fat measurements and have been used successfully for breast density measurements. DXA devices administer a much lower dose of radiation than traditional mammography, but are expensive and require a radiologist to interpret their output. Estimates of body composition using bioimpedance analysis are highly correlated (r=0.93) with estimates of body composition using DXA.

Bioimpedance assessment of body composition is used in over-the-counter scales, and there are over 50 FDA approved devices on the market indicated for the measurement of total body composition using impedance. Bioimpedance uses resistance as a measure of fat-free mass (FFM).

Having established the relevance of breast tissue density to diagnosis, prevention, and treatment of women suffering or at risk for breast cancer or related ailments, improved tests to determine this quantity (breast tissue density) are therefore useful. Breast density may be related to the amount of radio-opaque stromal, epithelial, connective, and blood vessels, etc. Percentage breast density can be related to the fraction of the breast that is comprised of radio-opaque tissue relative the amount of adipose (radiolucent) tissue which is closely related to the fat-free mass of the breast.

There presently lacks an apparatus or technique that can adequately measure breast composition directly and non-invasively using electrical impedance. In addition, there is a lack of systems and methods that can adequately provide the functionality of monitoring an individual's breast tissue composition over time. By way of background and illustration to set the stage and context of the present discovery and inventions, some related art is described below. The following discussions do not substitute for review and analysis of the references in the present context, and are not meant as legal characterizations of the references or features therein, but merely a simplified summary of the subject matter to which the references are directed, with some comment as to areas not sufficiently addressed by the references. Therefore, the references can be considered incorporated by reference for the background information therein, without the present inventor endorsing or agreeing with each statement made therein.

US Patent Application 2007/0293783A1 is directed to a system method of screening for breast by classifying patients into those that require further testing and those that do not using an electrical measurement. This method does not output a physiological measure of breast composition, and cannot leverage the existing clinical relating risk to breast composition. Its clinical utility and adoption are therefore limited. U.S. Pat. No. 6,122,544 is directed to a system and method for detecting and diagnosing disease by comparing the electrical impedance of homologous body parts (for example a left and right breast). The system does not report information about either breast's composition, and does not predict an individual's risk of breast cancer. In addition, the system requires a plurality of electrodes, the assessment of both breasts simultaneously, as well as a central hole, which can increase the cost of the system and its use.

US Patent Applications 2009/0306535, 2010/0049079, 2005/0203436 are directed to a system for measuring the condition of a region of epithelial or stromal tissue. The system calls for a connect between one of the measurement electrodes and the tissue under test (stromal and epithelial). Both of these tissue are subcutaneous and call for an invasive technique to make contact to them, such as those described in U.S. Pat. No. 7,630,759 and Application 2008/009764. These references relate to the notion of using the epithelial condition as a marker for changes from intervention. Invasive techniques described in the references include opening the nipple ducts through a variety of methods and establishing a direct electrical connection to the interior of the breast ductal system. This methodology also ignores non-epithelial and stromal tissue that contributes to the fat-free mass of the breast. Finally, the invention describes a methodology of measuring the ‘condition’ of epithelial tissue, not the amount which is considered to be the primary source of breast cancer risk.

US Patent Application 2006/0206271 describes a system body and body part measurement once again requiring a plurality of electrodes in contact with a plurality of predetermined body parts and requiring knowledge of the length of the body part to calculate its composition. Body part length is relevant to body parts that have length, such as a torso, leg, arm etc. It is difficult or impossible in some situations to define a breast ‘length’ that would lend itself easily to the present techniques.

U.S. Pat. No. 7,340,295 is directed to a system that calculates muscle mass. In a total body measurement device muscle mass is relevant. However as the breast does not have muscle mass, the system cannot be used to measure breast composition.

US Patent Application 2009/0171236 is directed to electrical bioimpedance as a biomarker for breast density. The reference generally measures a bioimpedance, limited to sub-epithelial impedance. The limits of the sub-epithelial impedance method, including invasive contact to the intra-ductal area are discussed above. Furthermore, this technique is aimed at indirectly determining mammographic breast density and does not provide direct measurement of breast composition. Mammographic breast density is a term whose meaning depends on the audience and context or technique so as to approximate a measure for actual breast composition.

It is known that certain pharmaceuticals are of use in treating or affecting a treatment of breast cancer. There also exists an accepted relationship between the breast density and the risk of developing breast cancer. For example, it has been demonstrated (J Natl Cancer Inst 2011; 103:744-752) that when there is a >10% reduction in breast density with the use of Tamoxifen there is a 63% reduction in breast cancer risk. In women with <10% reduction in breast density there is no reduction in risk.

When Tamoxifen or other endocrine therapies such as aromatase inhibitors are used, it has been shown that women whose breast density decreases in the first 8 to 12 months have a more than a twofold reduction in their risk of a cancer recurrence.

Tamoxifen (Nolvadex) is currently prescribed at 20 mg daily for 5 years by mouth. Raloxifene (Evista) is prescribed at 60 mg once a day with the optimum duration of treatment unknown. These risk reduction drugs come with side effects including increased risk of thromboembolic events. Tamoxifen use also carries with it an increased risk of endometrial cancer. Other side effects include symptoms of severe menopause. It is therefore preferable to minimize the amount of Tamoxifen to that which will result in the greatest risk reduction. However, it has not been appreciated if and how to employ such drugs or agents in the context of assessment of breast cancer risk in any useful or practical way. Other formulations of Tamoxifen and endocrine therapies drugs such as transdermal Tamoxifen (Tamo-Gel®) are available as well, and their side effects or efficacy could be optimized as described.

Therapy to reduce the symptoms of menopause such as hot flashes, fatigue, and vaginal dryness or atrophy consist primarily of pharmaceutical means of replacing the reduction in circulating estrogens and progestins. This type of therapy is called hormone replacement therapy (HRT). HRTs typically include proprietary mixtures of progestins and conjugated equine estrogens that are delivered orally, as a suppository, using subdermal implants, or with skin patches. Large studies have demonstrated an increase in breast cancer risk following the use of HRT. The type and duration of HRT has been linked to the increase in breast cancer. Studies have also shown an increase in breast density following an initiation of HRT which is commensurate with the type, intensity and duration of treatment. Of particular relevance are results which demonstrate that the change in density is a signal of a change in breast cancer risk with women experiencing the largest increase in breast density also experiencing the largest increase in risk for breast cancer.

Since mammography cannot be repeated frequently on a single patient due to the risks associated with exposure to ionizing radiation, the time scale of changes to breast density from hormonal changes such as endocrine therapy and HRT is unknown. However, studies with magnetic resonance imaging (MRI) have demonstrated that women on Tamoxifen can experience a measurable benefit in fewer than 3 months. MRI is too expensive to be used to longitudinally monitor women for changes in breast density. Therefore, a non-invasive non-ionizing low cost approach to tissue characterization is useful, including one that provides breast density assessment as well as diagnostic, predictive and therapeutic aids.

### SUMMARY

Aspects of the present invention are directed to a method for breast tissue characterization, comprising obtaining a non-invasive electrical measurement of breast tissue composition of a patient; and estimating a risk of future breast cancer corresponding to at least said non-invasive electrical measurement of said breast tissue composition.

Other aspects are directed to a system for determining a risk of future breast cancer in a patient, comprising a flexible multi-electrode applicator, having a first surface comprising a plurality of conducting electrodes for making non-invasive non-ionizing electrical contact with a skin tissue of a patient's breast at a corresponding plurality of locations thereon, and having a mechanical index for locating said flexible applicator on said breast with respect to an anatomical feature thereof; a measuring circuit in electrical communication with said plurality of conducting electrodes that generates an output signal corresponding to an electrical impedance of said breast; and a processor that receives said output of said measuring circuit and generates a result corresponding to a risk of future breast cancer.

Yet other aspects of the invention are directed to an applicator template for placement of electrodes on an external surface of a portion of a patient's body, comprising an insulating substrate dimensioned and configured to be applied to said portion of said patient's body; said substrate comprising a plurality of apertures permitting marking of a corresponding plurality of locations on said portion of said patient's body.

Yet other aspects of the present invention are directed to a method for delivering a treatment to a patient, comprising acquiring a first measurement of electrical impedance of a portion of the patient's body; initiating said treatment on said patient; waiting a determined length of time for initial results of said treatment; acquiring a second measurement of electrical impedance of a portion of the portion of the patient's body; comparing said first and second measurements; determining if said treatment changes a future risk of disease based on a predetermined criterion including changes in electrical impedance; and terminating said treatment if the change in risk of future disease in said patient is sufficient, and repeating the above steps if the change of risk is not sufficient or does not meet a preselected criterion, threshold or metric.

### BRIEF DESCRIPTION OF THE DRAWINGS

For a fuller understanding of the nature and advantages of the present concepts, reference is made to the following detailed description of preferred embodiments and in connection with the accompanying drawings, in which:

FIG. 1 illustrates an exemplary apparatus for acquiring body composition measurements;

FIG. 2 illustrates an exemplary block diagram of some components of a simplified patient interface unit (PIU);

FIG. 3 illustrates a block diagram of an exemplary system connecting the user interface module with other modules of the system;

FIG. 4 illustrates an exemplary block diagram of a MEU;

FIG. 5 illustrates an exemplary arrangement for clinical application of the present system and method;

FIG. 6 illustrates an AC constant current generator that uses a feedback system;

FIG. 7 illustrates the use of a voltage source and a large output resistor in a series circuit;

FIG. 8 illustrates an exemplary circuit receiving the output from the differential voltage measurement device;

FIG. 9 illustrates the relationship between the measured impedance (in Ohms) and the breast density;

FIG. 10 shows a typical relationship that may be derived between average measured impedance and BiRADS breast density;

FIG. 11 illustrates exemplary finite element (computed) solutions of the density in a given region of breast tissue;

FIG. 12 illustrates a comparison of measurements on a left and a right breast;

FIG. 13 illustrates exemplary placement of a pair of electrical contacts on the outer surface of a female breast;

FIG. 14 illustrates a pair of contacts being disposed on a circular applicator in a concentric fashion;

FIG. 15 illustrates placement of second sets of electrodes on parts of a body;

FIG. 16 illustrates an exemplary patient (breast) applicator and set of contacts fixed to the applicator;

FIG. 17 illustrates a bottom view of an exemplary patient applicator;

FIG. 18 illustrates the top side of the patient applicator;

FIG. 19 illustrates an exemplary tetrapolar measurement device;

FIG. 20 illustrates an applicator with a connector;

FIG. 21 illustrates another embodiment of a patient applicator template;

FIG. 22 illustrates another exemplary template applicator;

FIG. 23 illustrates steps of a method 990 for computing a future risk of a disease based on non-invasive non-ionizing measurements;

FIG. 24 illustrates exemplary steps of a method 1000 for monitoring the effect of therapy on breast composition and therefore breast cancer risk; and

FIG. 25 illustrates another method 1100 in which a women initiating hormone replacement therapy (HRT) such as oestrogen, progesterone or combination therapy is monitored.

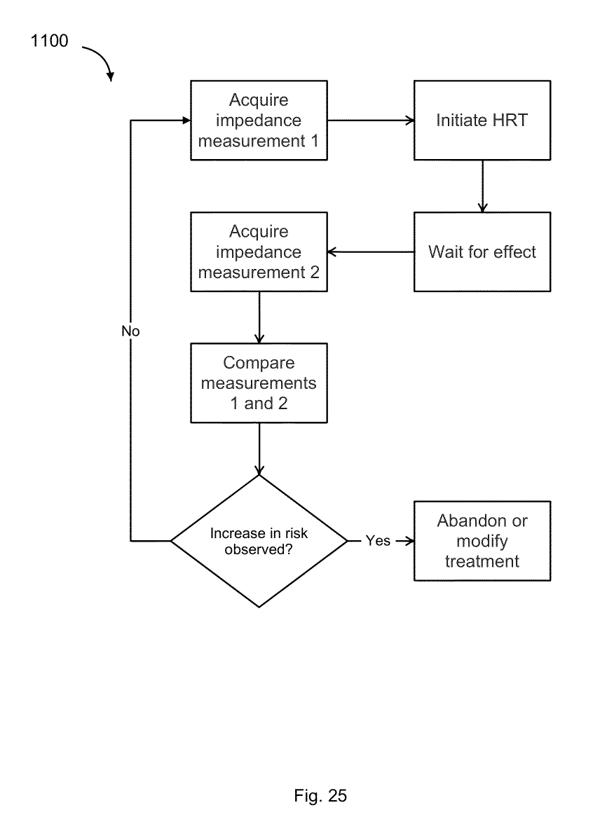

### DETAILED DESCRIPTION

Bioimpedance uses the fact that adipose tissue has a higher electrical impedance than fat-free mass. Electrical Impedance is defined as the ratio of V/I where V is the phasor voltage measured on an object and I is the phasor current passing through the same object. To measure electrical impedance at a single frequency one can inject a current of a known amplitude and phase angle and measure the voltage or introduce a known voltage and measure the resulting current. The magnitude of the ratio of the voltage and current is the magnitude of the electrical impedance, the real component of the ratio of which being the resistance and the complex component of which being the reactance. Those skilled in the art could appreciate from basic circuit laws that an electrical current, which takes the path of least resistance or impedance will travel primarily through fat-free mass. As more fat free mass is added to a pathway of the same length, it is as if there are more parallel electrical connections which results in a lower overall impedance. Therefore, the more fat-free mass (in a given length segment) the lower the segment's impedance.

The techniques of measuring bioimpedance are well known to those skilled in the art as is the advantage of a four electrode system—a four electrode system is generally used to measure the electrical impedance of biological tissue due to high contact impedance. Two electrodes are used as a current source/sink pair, and the remaining two are used to measure a differential voltage. The two voltage measurement electrodes are generally attached to a high impedance differential amplifier so that no current is drawn from these electrodes and there is therefore no significant voltage drop across them. Since voltage is not measured on the current source and sink electrodes, the voltage drop resulting from the contact impedance does not influence the measurement of impedance.

As discussed above, it is useful to measure tissue composition because tissue composition may have predictive power with regard to a person's risk of developing a disease and may help determine whether the person needs additional screening tests. Additionally, longitudinal measurement of tissue composition may also enable monitoring of changes in tissue composition that would correspond to changes in disease risk.

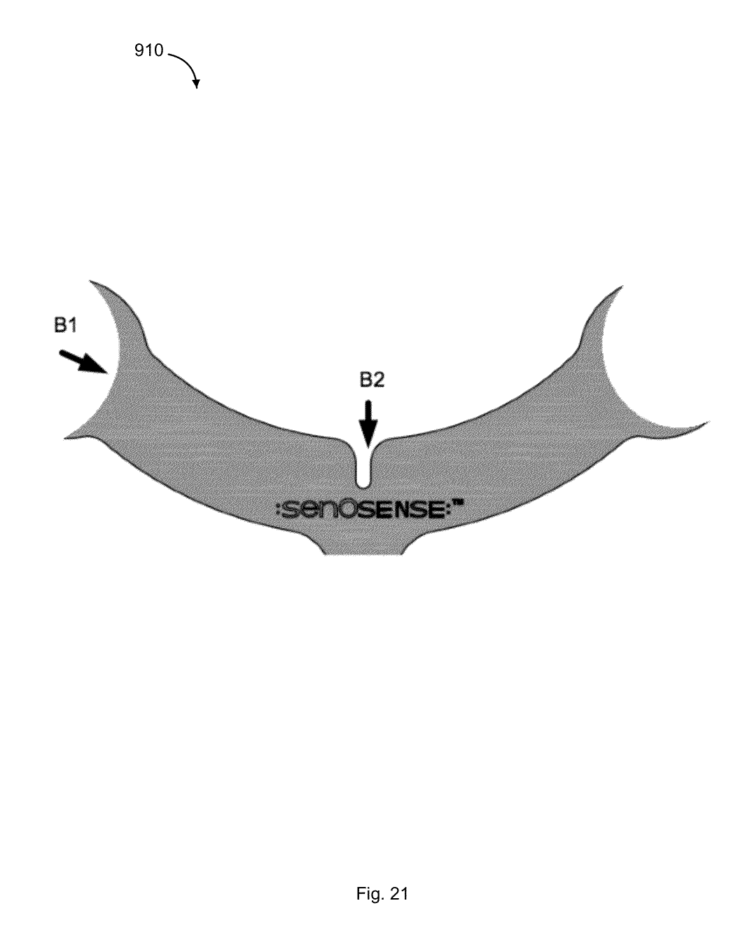

As shown in FIG. 21, the electrical impedance of adipose tissue has been determined to be substantially higher than that of dense glandular tissue. It is also well known that the amount of glandular tissue in a breast corresponds to future disease risk. In this disclosure a future risk of disease and a risk of future disease contraction are expressed interchangeably and relate to the probability or propensity to contract a disease at some future time, which can be expressed in metrics of lifetime risk, ten year risk, five year risk, and so on.

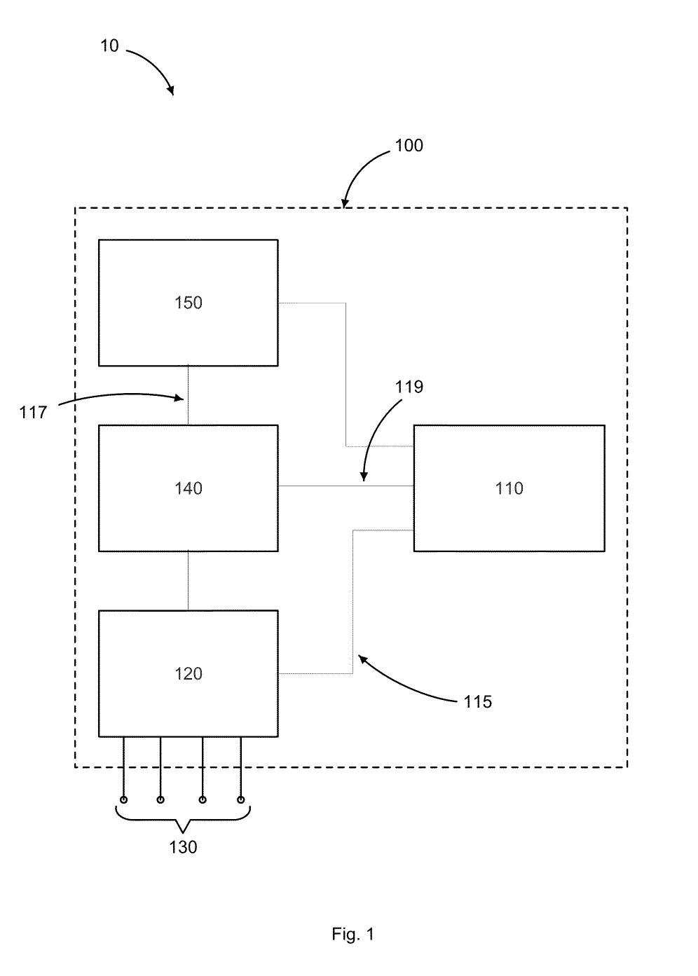

FIG. 1 illustrates an exemplary apparatus for acquiring body composition measurements (e.g., breast density measurements) and converting them to risk information (e.g., the risk of developing a disease such as breast cancer at some future time). According to an embodiment, the system 10 includes a main electronics unit (MEU) 110 and a patient interface unit (PIU) 120 coupled through a connection 115, which may be an actively shielded cable. The various parts of the system 10 may be housed in an enclosure 100 or may alternatively be housed in separate enclosures allowing independent positioning of the patient interface unit 120 onto a part of the patient's body as will be described below.

The patient interface unit 120 includes a plurality of connections 130 between the device and a part of a patient's body. The connections 130 can comprise electrical sensors or couplings or electrodes to make a measurement of a parameter of the body part with which they are placed in contact with. It is not essential to make direct physical contact with the body part, and in some instances the measurements by the connections 130 can be made without direct contact with the patient's body. Instead, capacitive or coil-based contacts may be employed. Generally, the patient interface unit 120 and the connections 130 are non-invasive and do not penetrate the patient's skin or body. However, a combination of the present device with other devices, including invasive devices can be envisioned and used by those skilled in the art as needed.

In an embodiment, analog measurement signals include a current source electrode and a current sink electrode as well as a pair of voltage measurement electrodes. These four electrodes may be applied to the surface of the skin of an organ of interest such as the human breast, as will be discussed in more detail below. The MEU is adapted to determine an electrical impedance of the organ being tested, which impedance is represented by a ratio between the voltage measured by the two voltage measurement electrodes and the current passing through the two current electrodes.

The patient interface unit 120 may be coupled to an impedance module 140 to enable measurement of electrical impedance of a portion of a patient's body. A user interface module 150 facilitates the input and output of information from the rest of the system. Those skilled in the art appreciate that the arrangement of the individual modules shown and described herein may be accomplished differently than the one depicted in this exemplary embodiment. In a general embodiment, the main electronics unit (MEU) can be used to monitor and control the operation of the other units (e.g., 120, 140, 150) and can incorporate custom or known microprocessor based circuitry including machine readable instructions that execute on the MEU when in operation.

In some embodiments, analog instrumentation is provided in impedance module 140 that connects both a patient interface sensor in the PIU 120, and a user interface 150. The resulting impedance can be transmitted over a communication interface 117 such as a USB connection to the user interface 150.

As stated earlier, any or all of the components mentioned above may be disposed within a same housing 100 or separate housings as suitable for a given application. For example, the user interface module may reside in a separate physical location, and may include interface connections and connectors to exchange data with any number of known and useful interface options. Likewise, the patient interface unit 120 may be separated from the rest of the system and can be hand held or remote therefrom, especially in the connections 130 leading to the patient measuring portions of system 10.

It can be appreciated that system 10 employs various conducting connections and signal conduits or bus work to carry information in analog and/or digital form between the various components. The communication signal paths may include an isolation system providing electrical isolation between the input connection of the user interface 150, which can be achieved through devices such as the ICoupler from Analog Devices, or optical isolators such as the ISO from Texas Instruments. If the interface to the user interface module 150 contains power such as an industry standard USB 1.0 or 2.0 interface, the impedance module 140 can be powered from this interface. The USB power can be isolated using an isolating DC/DC converted such as Texas Instrument TPS 55010. Alternatively, a separate isolating power supply can be used. Isolating the signals and power supply to the impedance module enable a further alternate embodiment in which the impedance module 140 can be safely connected to a conventional computing device (e.g., a PC), and provide impedance measurements to software installed on that PC which can perform risk calculations and other operations using the measured impedance data. A network connection and interface are optionally used to allow the apparatus to receive data from and provide data to another machine on said network, whether local or remote.

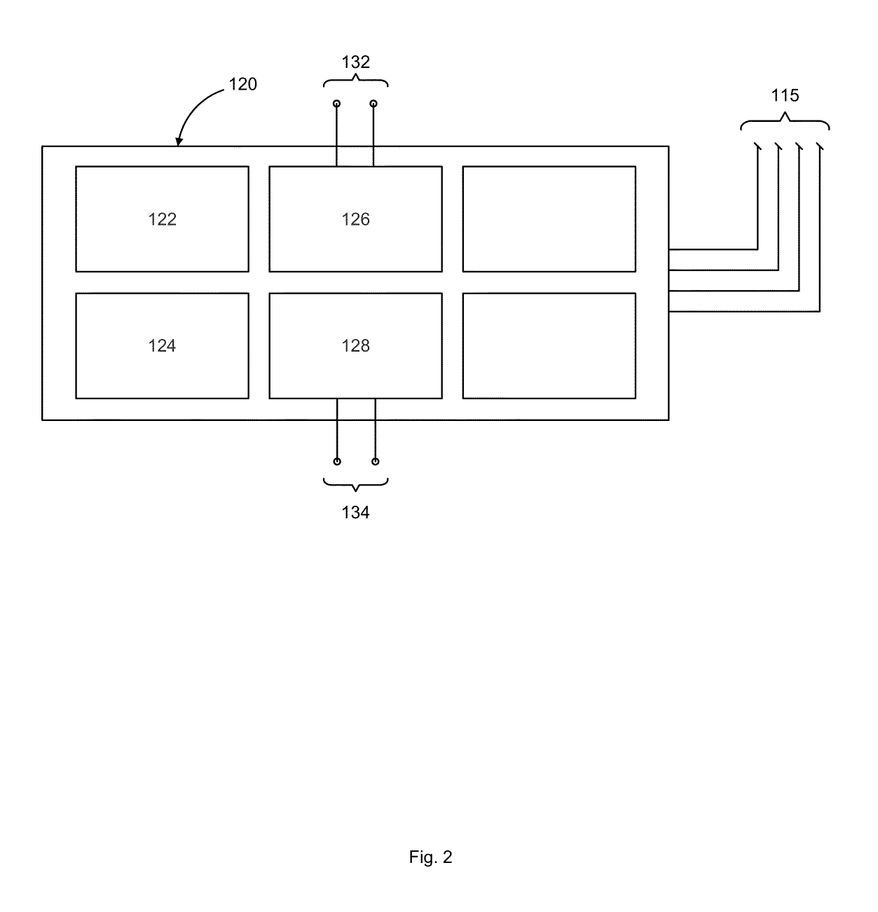

FIG. 2 illustrates an exemplary block diagram of some components of a simplified patient interface unit (PIU) 120. For example, the PIU 120 may include in some embodiments: a digital to analogue (D/A) converter 122, an analogue to digital (A/D) converter 124, a differential voltage measurement device 126 coupled to a pair of voltage sensing electrodes 132 and constant current generator 128 coupled to a current source-sink pair of electrodes 134. Amplifiers and bridge circuits and other components of the patient interface unit 120 may also be provided as required by a specific design and as would be desired by those skilled in the art. Further details of the mechanical design of an exemplary PIU and electrode placement apparatus will be described below.

We discussed the inclusion of a user interface in the present system. In some aspects, the user interface can also be used to deliver a breast cancer risk questionnaire allowing the data required for other risk models to be entered and combined on a single device. The user interface module 150 can optionally be connected to a printer directly or through a printer to provide a hard copy record of the breast density assessment and resulting risk assessment alone or in combination with other factors. The questionnaire module queries the patient or operator for information relative to the patient's risk of disease. The risk calculation module converts the measured impedance, as described in detail below, into risk information, and combines that risk information with the risk information recorded by the questionnaire module also described in detail below. The report module presents the risk information on the screen and enables a hard copy output to be generated. The optional database module stores impedance and risk information per patient so that changes can be tracked and compared between visits. The user interface can also be used to deliver a breast cancer risk questionnaire allowing the data required for other risk models to be entered and combined on a single device. The user interface module can optionally be connected to a printer directly or through a printer to provide a hard copy record of the breast density assessment and resulting risk assessment alone or in combination with other factors. The user interface can consists of an interface module I4, a questionnaire module I3, a risk calculation module I5, an output module I6, and an optional database module I7. The interface module communicates with the impedance module H1 to receive impedance measurements.

A disease risk analyzer or module may include hardware and software for calculating a future risk of a disease based on inputs to the risk analyzer. For example, a patient interface may provide an output signal or data representing a characteristic of the patient's body or an organ of the patient. In one instance, this can be a density of the human breast. Using this information, and optionally other physical or historical information, an output signal or data representing future risk of disease (e.g., breast cancer) can be determined. The patient interface provides a mechanism whereby measurement signals can be coupled from the tissue composition measurement components to the patient without introducing any material or test devices into the patient's body and without using ionizing radiation. In the example used earlier, the resulting impedance as determined by the tissue composition measurement components can be processed by the risk management component of the system to output information on the patient's future likelihood of developing breast cancer. The risk evaluation process can output one or more of the patient's 5 year, ten year and lifetime risk of developing breast cancer, which are stored or presented on an output device (display, printer, etc.)

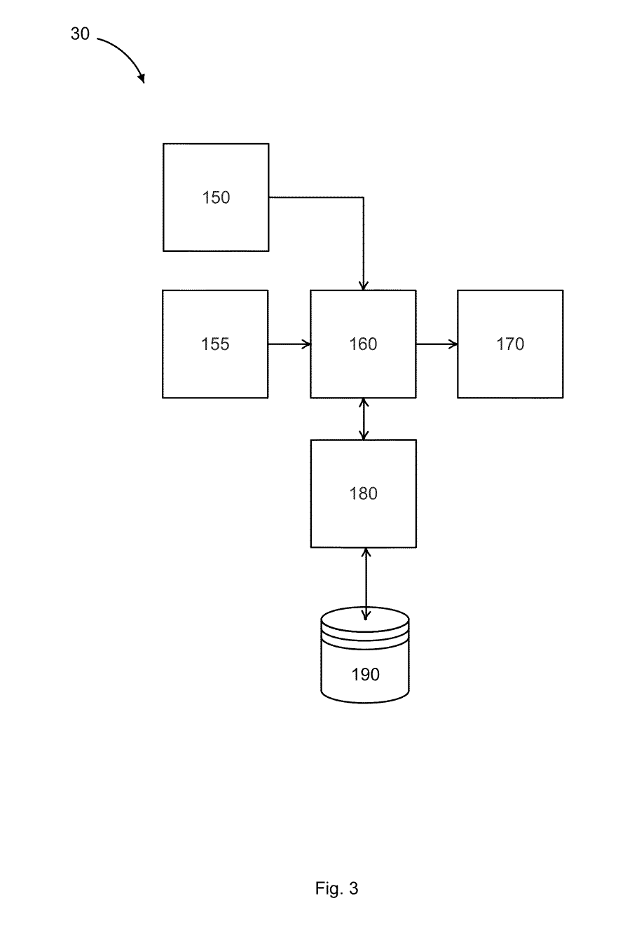

FIG. 3 illustrates a block diagram of an exemplary system 30 connecting the user interface module 150 with other modules of the system. Here, the user interface module 150 is coupled to a risk calculation module 160, an output module 170, and an optional database module 180 that may be coupled to a database 190. The interface module 150 communicates with the impedance module 140 discussed before to receive impedance measurements. The risk calculation module 160 converts the measured impedance, as described in detail below, into risk information. The output or reporting module 170 presents the risk information on a screen and enables a hard copy output to be generated. The optional database module 180 stores impedance and risk information per patient so that changes can be tracked and compared between visits. The database 190 can also be used by these or other components of the system 30 to retrieve historical data and retrieve data from a look up table (LUT). Other embodiments may employ a user interface, such as a computer based graphical user interface to enable a provider to interact with the apparatus of the invention and to enter information into and receive output from the apparatus.

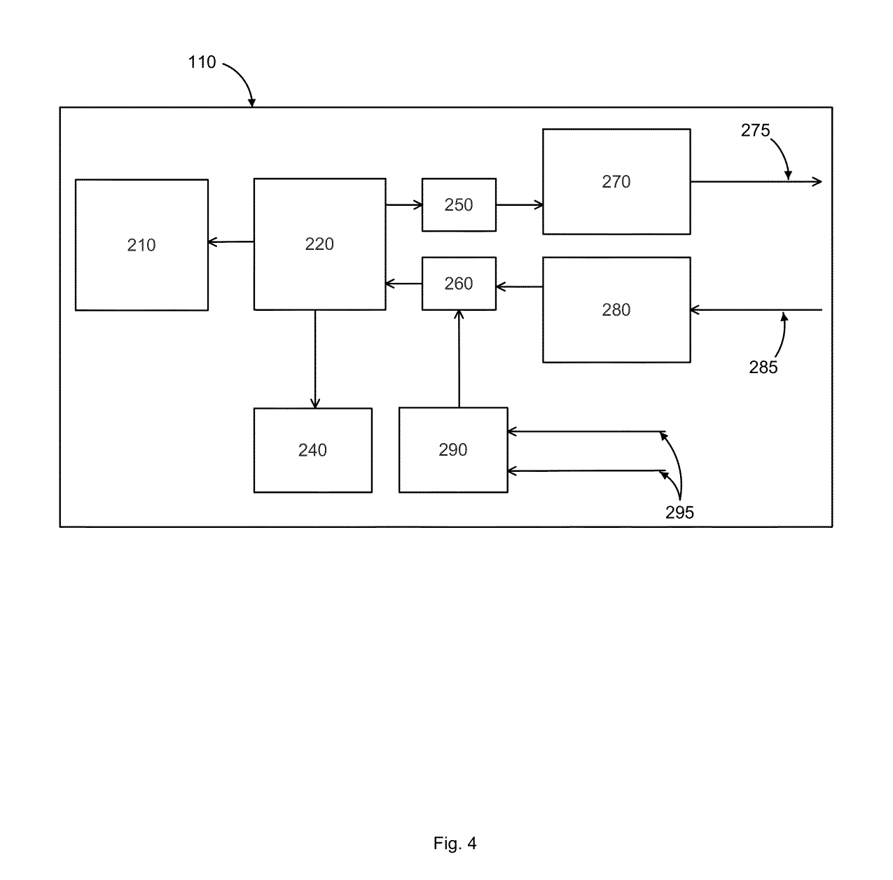

FIG. 4 illustrates an exemplary block diagram of a MEU 110. MEU 110 includes a microprocessor 220 that executes program instructions and controls the overall operation of MEU 110 and other components. The MEU 110 operates on analog measurement signals from the patient interface unit 120.

In operation of the present exemplary embodiment, voltage input signals are received at 295 by differential voltage measurement device 290 which can be an instrumentation amplifier like the Texas Instruments INA-128 or other circuit, e.g., comprising operational amplifiers. An input return current signal at 285 is further received by reference signal generator 280. In some embodiments, a reference signal generator 280 may be used to control the potential on the returning electrode to minimize common mode of the measured voltage signals. Outputs from said differential voltage measurement device 290 and said reference generator 280 are provided to an analog-to-digital (A/D) converter 260, which in turn provides a digital output to microprocessor 220.

Microprocessor 220 executes stored instructions and operates on input signals such as from A/D converter 260. Microprocessor 220 provides outputs to a user interface, for example to a display screen 210, or even to a printer 240. Microprocessor 220 also provides a digital output signal to digital-to-analog (D/A) converter 250, which is fed to an AC constant current generator device to produce a current output at 275. Current output 275 and current return 285 are related, and consistent with the present discussion, inform the determination or measurement of a characteristic of a patient's body composition. For example, the current signals at 275, 285, along with the voltage signals at 295 may be indicative of an electrical, or electro-mechanical attribute of a patient's body or an organ of a patient, e.g. a female breast.

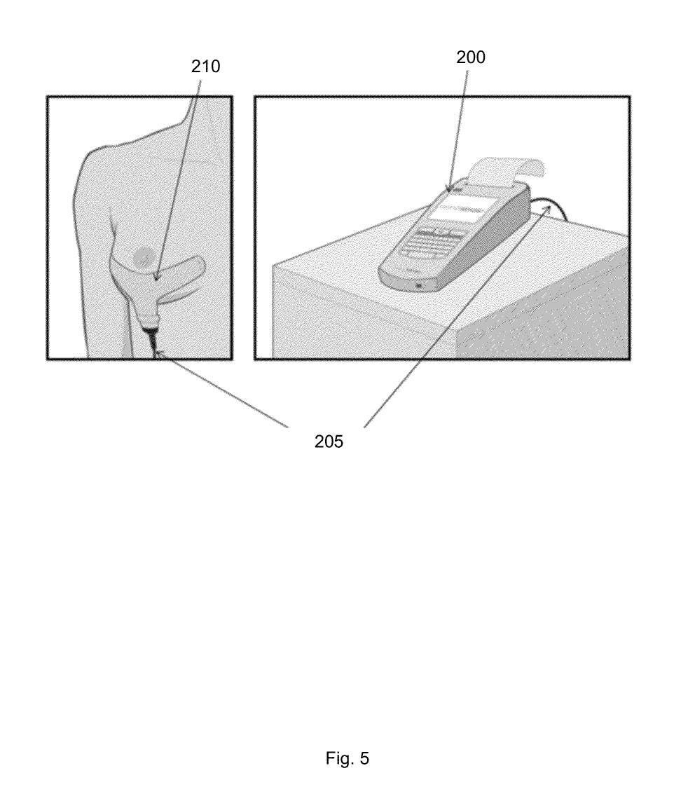

FIG. 5 illustrates an exemplary arrangement for clinical application of the present system and method. The frame on the right shows generically how a portable housed apparatus 200 can be provided with various user interface features and contain processing circuits and instructions to carry out the functions described herein. The device 200 may be powered by an electrical source (A/C or D/C battery power) and may be hand held. A patient (breast) applicator 210 is applied to the surface of a breast of a patient for measuring the electrical impedance of the breast tissue of the patient. Power and signals are carried between the breast applicator 210 and the apparatus 200 by a power/signal cable 205.

Alternatively, the parts 200 and 210 of the system can be in wireless communication with one another (e.g., using Bluetooth®, infra-red, radio frequency or other communication). In yet another embodiment, the main unit 200 may be separately available from the breast testing applicator 210, while the applicator 210 may contain the required testing components and a memory to hold data from a test until the applicator is returned to the main unit 200 and coupled thereto. In this way, one main unit 200 can be provided in a clinic while several (or many) breast testing applicators 210 can be deployed in a clinical facility or in the field (outside the facility) to test a corresponding number of patients. Once tested, the applicators can be returned to the facility and connected to the main unit for readout or risk calculation based on the test results.

Having described the present system at a generalized level, we now turn to illustrative examples of the construction and operation of the patient testing parts of the system, and in particular, to an example where the salient measured feature is the electrical impedance of the tissue of interest using non-ionizing and non-invasive techniques and devices. Then, we will describe exemplary arrangements and configurations of an applicator suited for measurement of impedance of the human (female) breast. As stated before, the present illustrative examples should not be considered exhaustive or limiting. But rather, they are provided for clarity of presentation of the invention. Those skilled in the art will appreciate alternative designs and configurations and alternative uses of the present system and method upon review of this disclosure. All such permutations are intended to be within the scope of the invention and the appended claims.

We now examine a method for measuring electrical impedance of tissue from conducting contacts or electrodes placed upon the skin of the patient in an area of interest (e.g., a breast, abdomen, etc.). A constant current generator and voltage sensing points are employed, the relationships between which are used to deduce the impedance of the tissue on which they are placed.

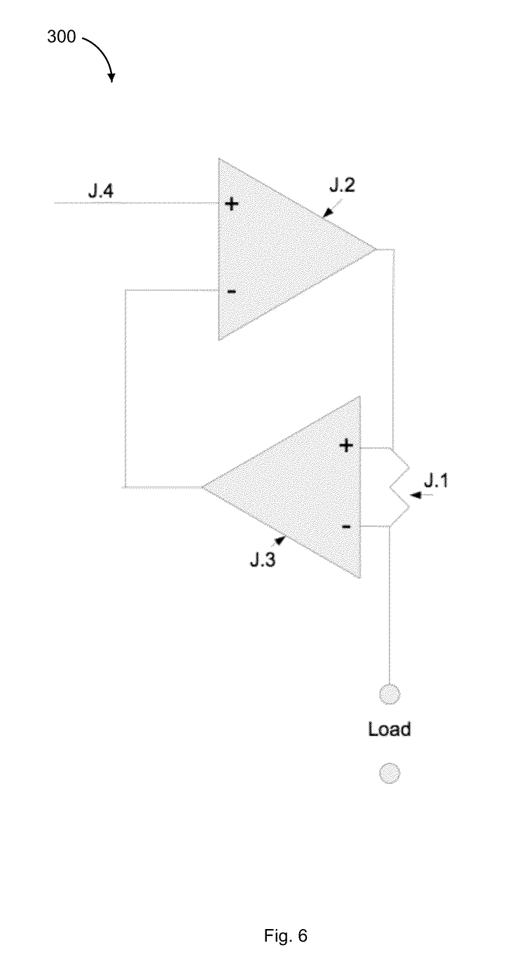

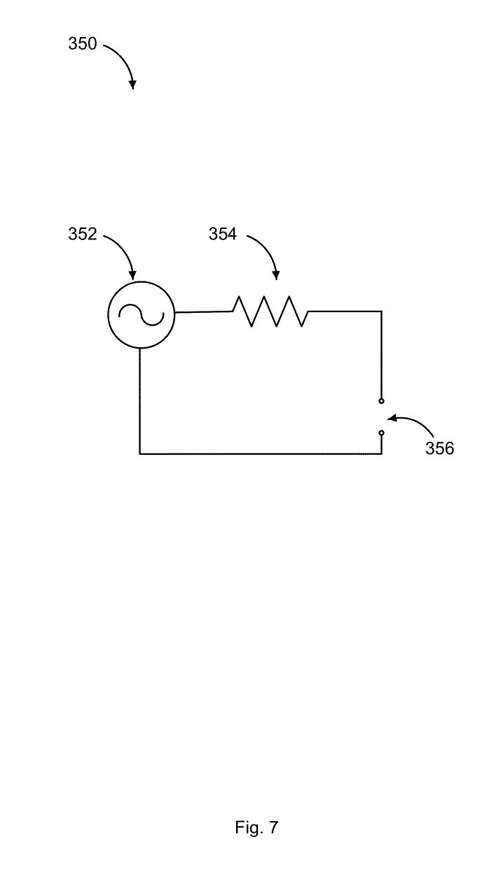

FIG. 6 illustrates an AC constant current generator 300 that uses a feedback system. The current is measured by measuring a voltage drop across a resistor (J.1) using an instrumentation amplifier such as the INA 128 (J.3). The resulting output current is compared to the desired signal as output from the D/A (J.4) using an operational amplifier (J.2) such as Linear Technology's LT1006 and the output voltage is adjusted until the difference between these signals is nulled and the current delivered corresponds to the D/A output 250. The relationship between the D/A output and the output current is set by the resistor J.1, and the gain of the instrumentation amplifier using the following relationship I=Vin/(R*G) where R is the resistance and G is the gain of the instrumentation amplifier. FIG. 7 illustrates the use of a voltage source 352 and a large output resistor 354 in a series circuit. The current change from a change in load 356 is small so long as the large output resistor 354 is much larger than the impedance under examination connected at the load terminals.

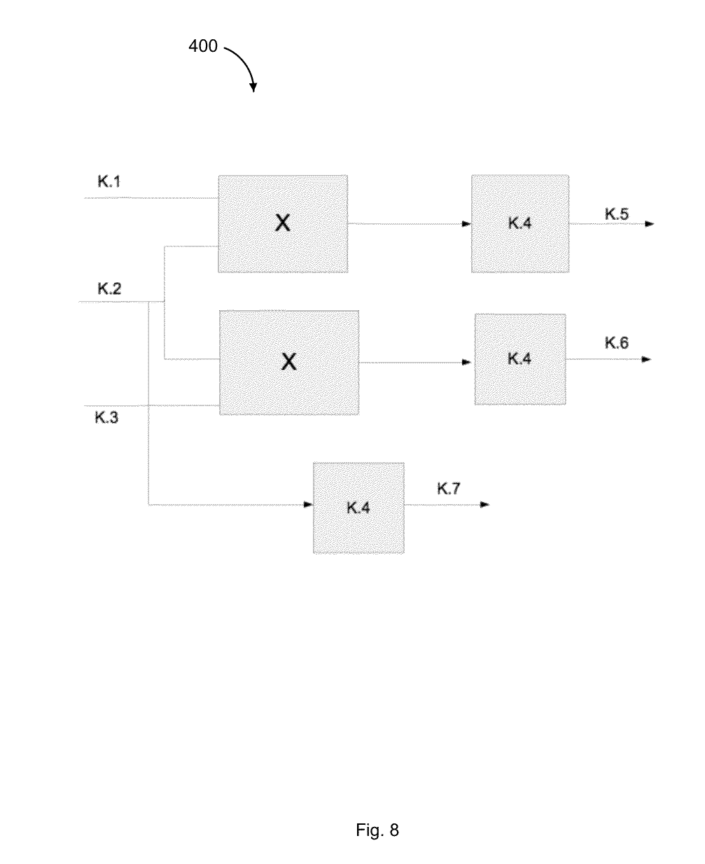

FIG. 8 illustrates an exemplary circuit 400 receiving the output from the differential voltage measurement device 290 that is converted to an impedance by an electronic synchronous detector. The circuit 400 employs a signal in phase with the injection current (K.1) and a quadrature signal (90 degrees phase shifted) K.3 square wave each independently multiplied with the differential voltage signal (K.2) using a multiplier circuit (K.7) such as the AD539. The resulting products can be integrated using an electronic integrating circuit (K.4) which can consist of an RC filter or active integrator to produce in-phase (K.5) or quadrature (K.6) impedance components known as resistance (R) and capacitive reactance (X_{c}) respectively. These components can be digitized using an A/D converter and converted to impedance magnitude Z using the relations Z=SQRT(R²+X_{c}²). When the components are digitized the duration of acquisition can be selected based on a trade off between speed of measurement and noise rejection. Alternatively, and also shown in FIG. 8, the magnitude of the impedance Z (K.6) can be obtained by directly integrating the voltage output of the differential voltage measurement device 290 using an integrator (K.4) where the time constant determined by the product of R and C can be selected to optimize between acquisition time and noise rejection. Z, R, and/or X_{c} can be reported to the operator and provide information on body part composition. Each of R, Z, and X_{c}, can be digitizes by an A/D and processed into risk information via the microcontroller as discussed elsewhere.

In another embodiment, the differential voltage signal output from measurement device 290 is digitized directly using an A/D operating at a sampling rate of more than twice the current injection AC frequency. Higher sampling frequencies will enable greater to signal to noise ratio and/or shorter sampling times. The resulting voltage signal can then be multiplied against an in-phase and quadrature signal stored in memory to obtain R, X_{c}, and Z.

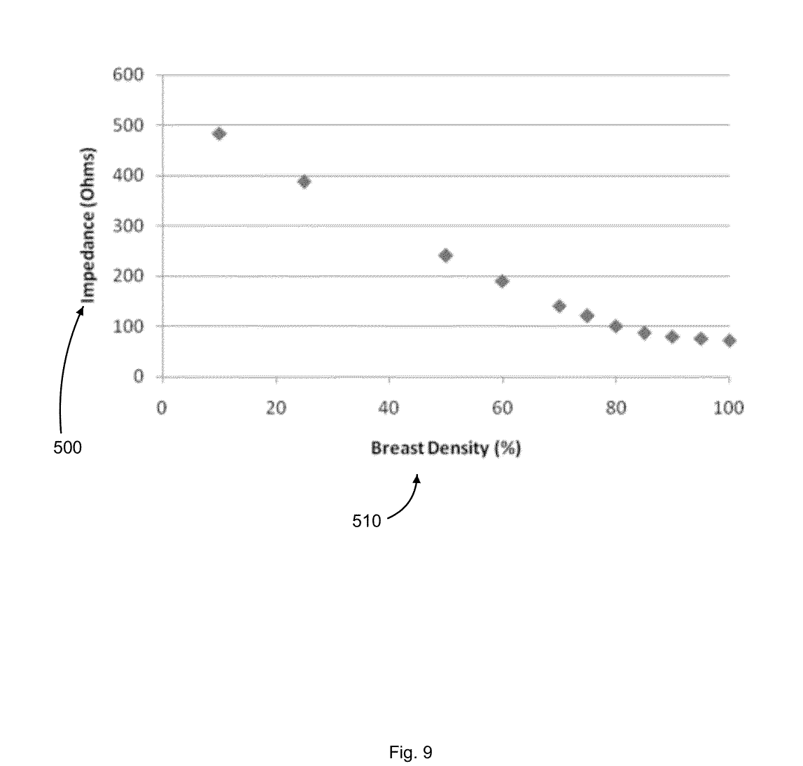

FIG. 9 illustrates the relationship between the measured impedance 500 (in Ohms) and the breast density 510 (as a percentage of a baseline) using the present system and method. This exemplary illustration depends on a number of algorithmic and design factors, and is not provided by way of limitation. The results of one or many such measurements can be developed into a lookup table or a polynomial or other mathematical expression that can be used to infer or determine the density of the breast tissue from a measurement of its impedance.

Breast composition as measured in each region of the breast using an apparatus such as those described above, or over the whole breast. The results of the measurements can be related to a database of normals (patients representing the general population) to establish where that regional density is relative to expected regional or overall densities. This number can be reported as a percentile rank. A patient's rank score can be calculated as follows: Rank Score=Rank*100/# where Rank is the rank of that breast composition or impedance score when all patients are sorted, and # is the total number of subjects studied. Alternatively a patient's z-score where z=(patient impedance-average normal impedance)/(standard deviation of normal impedances) can be calculated relative to the normal population indicating how far a particular patient's impedance is from what is considered normal.

Alternatively, a threshold can be applied identifying whether the measurement indicates substantially increased risk. The threshold can be applied based on the size of the population that is desirable for additional follow up. It is noted that the use of impedance corresponds to all aspects of an impedance measurement including resistance, reactance, magnitude and phase, all of which can be used to compare a patient to a normal population.

Alternatively the impedance measurements can be translated into mammographic estimates or percent density using a table or graph of impedance measurement versus mammographic density as shown in FIG. 9. These results can be generated by creating breasts with different compositional properties and surface electrodes as described in this disclosure. Each region is assigned a conductivity, for example as shown in Table I below:

TABLE I

| Region | Conductivity (S/m) |
|---|---|
| Electrodes | 1e+6 |
| Fat | 25 |
| Glandular Tissue | 200 |
| Skin | 25 |

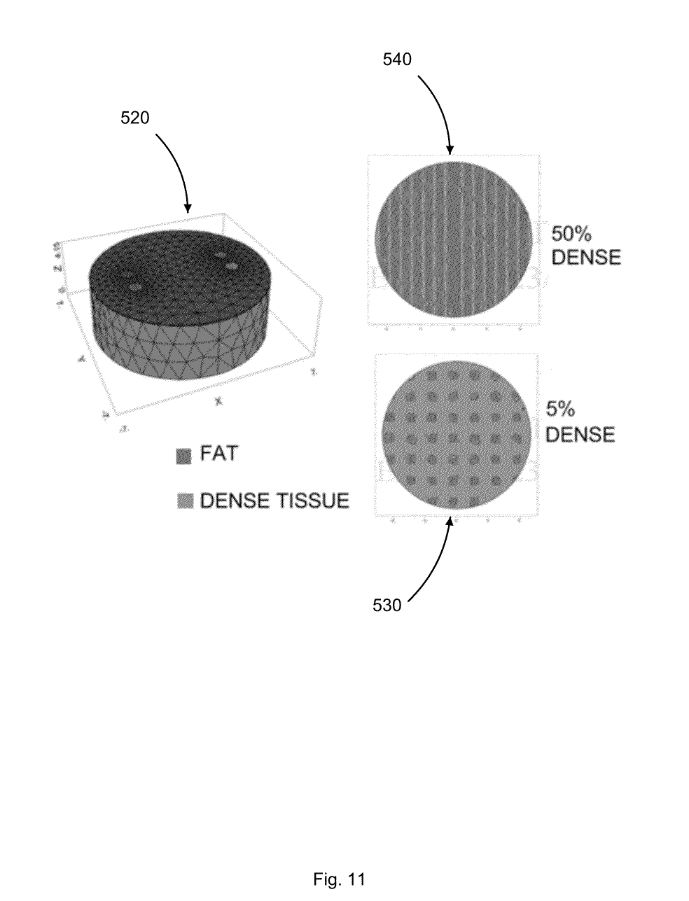

FIG. 11 illustrates exemplary finite element (computed) solutions of the density in a given region of breast tissue for two different density patterns. A finite element solver such as FlexPDE from PDE solutions can be used to solve the surface potential when a fixed constant current is injected into the surface through electrodes by solving the partial differential equation Div(−s(Grad(V)))=0. The current density is calculated as −s*Grad(V). Where s is the conductivity in S/m, V is the Electrical potential (Voltage) and Grad and Div represent Gradient and Divergence operators respectively. By dividing the voltage solved by the total current injected through an electrode, one can calculate the surface impedance. To convert from a measured impedance one would identify the measured impedance on the graph that was measured and use the graph to select the corresponding percentage breast density.

Alternatively the impedance measurements can be translated into a volumetric breast density as acquired by other means such as MR, mammography or DXA using data acquired from a clinical study. Data points may be derived from a clinical study in which human readers or automated software such as the Volpara software are used to measure breast density on the same women who have and their impedance measured.

Alternatively the impedance measurement can be translated into a relative risk versus the normal population of that breast density based on studies either acquired on the impedance technology or first translating the impedance measurement to a mammographic density estimate and using published relative risk ratios for the calculated density. In the latter case one can use published data such as that provided in Boyd et al. N Engl J Med 2007; 356:227-36. The relative risk for 0-25%, 25-50%, 50-75%, and 75%+ are presented as an example result. To obtain an individual's relative risk of breast cancer, one would measure their impedance as described in the related disclosure, use one of the methods described above to obtain a breast density, and use one of the published data sets such as the example above to convert that density to a relative risk of developing breast cancer.

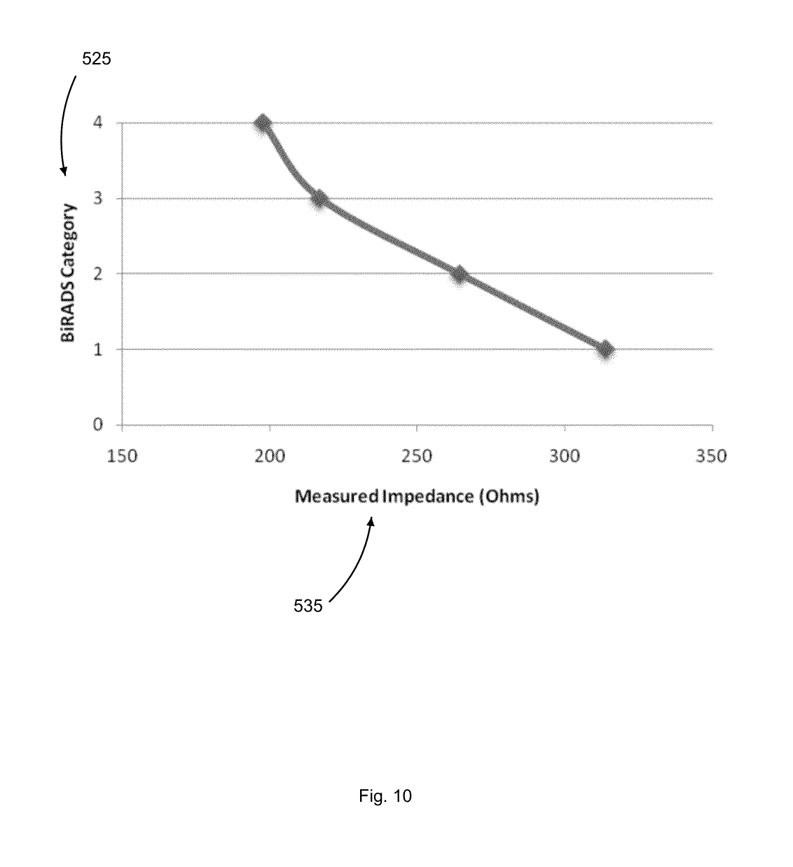

In addition, one can also generate a conversion between measured impedance and BiRADS breast density categories I-IV by having readers assess the density of the mammogram of a patient who has also had their breast density measured. FIG. 10 shows a typical relationship that may be derived between average measured impedance and BiRADS breast density, which may be used (or an equivalent or similar relationship used) by the present system and method. The relative risk can then be calculated using published data of BiRADS category density such as that published in Breast Cancer Research and Treatment (2005) 94: 115-122. In this method the relative risk calculated for a patient with BiRADS I, II, III, & IV is 0.59, 1.00, 1.41, 1.94 respectively.

Alternatively the impedance measurement can be translated to a five year, other duration, or lifetime risk of breast cancer based only on the increased risk of breast density and the patient's age. One can use available data on breast cancer risk by age group such as that published by the US Center for Disease Control (CDC) at http://www.cdc.gov/cancer/breast/statistics/age.htm, reproduced below in Table II.

TABLE II
Percent of U.S. Women Who Develop Breast Cancer over 10-, 20-,
and 30-Year Intervals According to Their Current Age, 2005-2007

| Current Age | 10 Years | 20 Years | 30 Years |
|---|---|---|---|
| 30 | 0.43 | 1.86 | 4.13 |
| 40 | 1.45 | 3.75 | 6.87 |
| 50 | 2.38 | 5.60 | 8.66 |
| 60 | 3.45 | 6.71 | 8.65 |

The relative risk of breast density can then be multiplied against the risk from published literature to produce the patients personal risk of developing breast cancer in a time interval.

In another embodiment the impedance measurement can be combined automatically on the device with other risk factors such as age, parity, hormonal history, BMI, genetic status, family history, alcohol consumption, and race to produce a more detailed overall five year, other duration, or lifetime risk of developing breast cancer using standard risk models such as BRCAPRO, Claus model, Gail model, or Tyrer-Cuzick. To modify these models, the risk outputted from the model can be multiplied by the relative risk determined by the measurement of mammographic density as described above.

In another embodiment the impedance measurement device prompts the user for information that can be input into the models such as BRCAPRO, Claus model, Gail model, or Tyrer-Cuzick and automatically reports the aggregate risk information.

In other embodiments the automatic combination of risk models such as BRCAPRO, Claus model, Gail model, or Tyrer-Cuzick is done with other methods of breast density estimates including sub-epithelial impedance, e.g. AP12316032, mammographic density as assessed by a reader or with CAD, MRI assessment of density, or infrared transillumination spectroscopy.

Note that the present system and method can be used on an organ of interest such as a human breast. That is, the present technique does not require comparison of one organ against another such as a comparison of the left and right breasts of a patient. The present measurements are made on the organ under investigation (e.g., a patient's left breast), but can be repeated for reference or comparison or averaging purposes on another organ (e.g., the patient's right breast) but this is not required to obtain the results discussed herein.

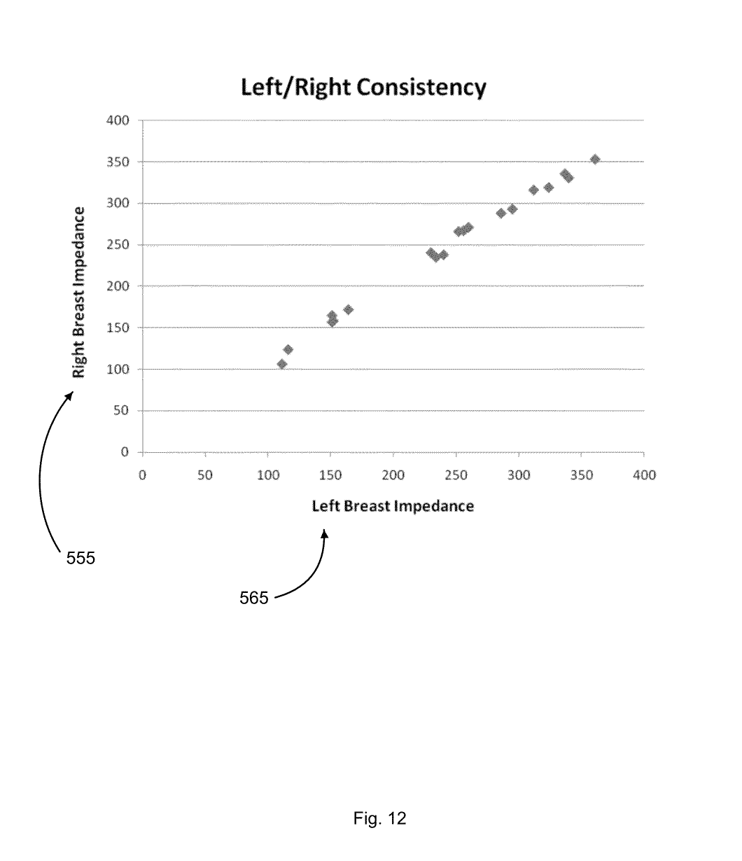

With that said, there may be useful information in checking the left/right consistency of a patient's breast density or electrical impedance measurements as illustrated in FIG. 12, plotting the impedance of a right breast sample 555 against that of a corresponding left breast sample 565. FIG. 12 shows a comparison between independently derived right and left impedance based breast composition measurements. Since the relative placement of all patient contact points is fixed, the measurements between left and right breast are highly reproducible, with a correlation coefficient over 0.98 enabling detection of a small change in tissue composition.

The following discussion introduces the applications and uses of the present system with exemplary focus on applications to the study of breast cancer and breast density factors in human patients. Design considerations, especially for the configuration of the patient or breast organ applicator will become more apparent upon review of the examples below.

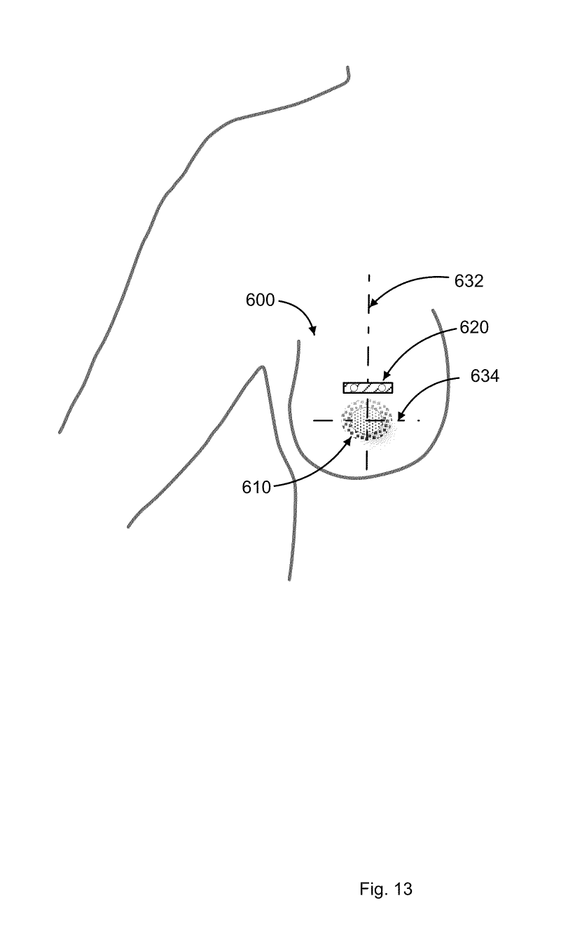

FIG. 13 illustrates exemplary placement of a pair of electrical contacts 620 on the outer surface of a female breast 600. The contacts 620 may be fixed with respect to one another. The contacts 620 may be positioned on the breast 600 relative to an anatomical feature of the subject, e.g., the subject's nipple 610. Alternatively, an actual or imaginary line or lines 632, 634 may be employed to position the contacts 620. The contacts can be moved from one region of the breast to another, and from one breast to another to measure the local breast composition. The contacts 620 are shown disposed on a rectilinear, or linear, applicator body. However, this is not limiting.

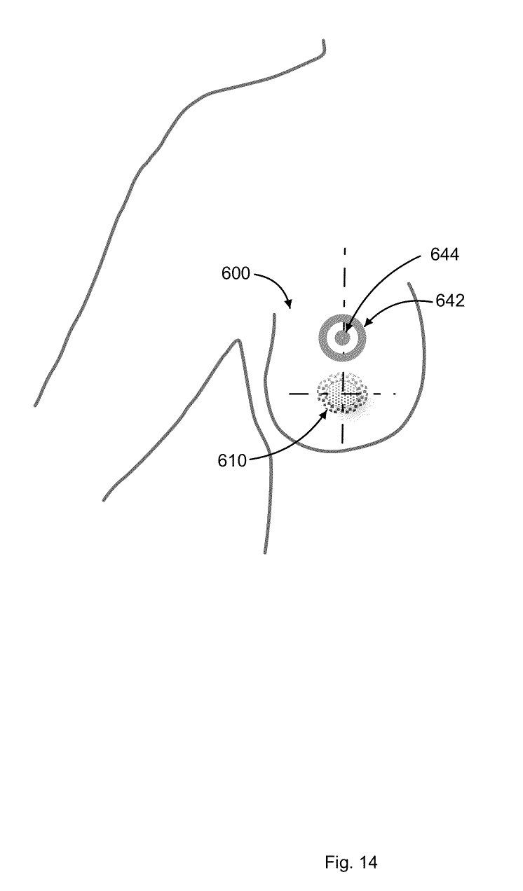

FIG. 14a illustrates a pair of such contacts 642, 644 being disposed on a circular applicator in a concentric fashion. The applicators to which the electrical contact points are attached may be of a thin and flexible nature, such as laminar, e.g., polyester, sheet stock, which allows the applicators to easily conform to the anatomy of the subject and organ under study. Also, the relative sizing, spacing and location of the contacts and applicators may vary to aid in localizing a measurement if that is desired. It is envisioned that applicators of various shapes and sizes, some of which are described below in more detail, may be employed depending on the clinical need and the anatomical dimensions of the patient under investigation.

As to manufacturing and electro-mechanical implementation of the present apparatus, it can be achieved by proper placement of the plurality of electrical sensors, contacts, or conduction points on either a flexible support member or applicator adapted for placement in proximity or contact with the surface of the body organ in question (for example the female breast), or a more rigid, handheld device. The handheld device can be used in combination with a flexible support member, attached by a cable, or in combination with another rigid device that is held by the patient or placed on an arm or leg.

The apparatus described herein may include four or more electrode pads on a patient applicator, as mentioned above, a first pair of which are used to carry an electrical current through the tissue under examination (one source and one sink), and a second pair of which are used to measure a differential voltage (electrical potential) there between. Together, these are used to determine the electrical impedance of the tissue under examination. The impedance is then used to determine a condition of the tissue, which can be correlated with or correspond to a clinically-relevant diagnosis or health metric or a risk of future incidence of a disease in a patient.

FIG. 14b illustrates a second set (pair) of contacts providing the return current path and voltage reference is held by the patient 660 or adhered to her body in a location remote to her hand such as the leg or abdomen 661. Changes in body composition in a specific region may indicate elevated risk of disease in that region. Alternatively a single large electrode can be used as the reference since its interface impedance can be small enough not to necessitate separate voltage and current contact. This single electrode can be applied on the body 662 or held by the patient 663. The impedance measured between a smaller pair of electrodes such as those shown in 620, 642 and 644 and a larger reference electrode or electrode pair will be substantially dominated by the smaller electrode pair. The current density is much higher closer to the smaller electrode pair, and the contribution of a tissue region to the overall impedance measured is determined by the current density in that region. Having a remote fixed return electrode allows the electrode pair to be moved around the surface of the body part of interest. The current density in the tissue not immediately under the moving electrode pair is similar between different placements of the moving electrode pair, and therefore the difference in impedance between different locations is driven primarily by the impedance immediately under the placed electrode. Furthermore, since the current will spread out as it enters the body, the current density in tissue remote from the moveable electrode is much lower and therefore its overall contribution to the measured impedance much less than the region immediately under the electrode. The two sets of electrodes can be connected through separate connections to the impedance module 140.

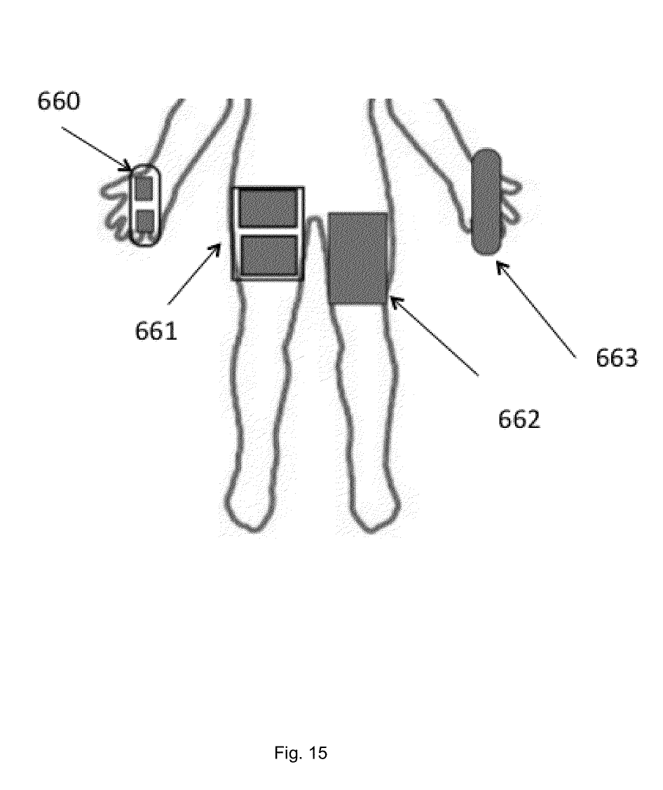

FIG. 15 illustrates an exemplary patient (breast) applicator 700 and set of contacts fixed to the applicator. This embodiment provides an integrated tetrapolar applicator comprising a thin flexible substrate 710, which may include an adhesive material to help hold it in place once it is positioned on the patient. The applicator device 700 has four conductive areas or conductive wells or electrodes 715 as described earlier to be in current and voltage contact with the surface of the breast. The applicator 700 can include a positioning notch 720 for alignment of the apparatus 700 with respect to the patient's nipple. The thin flexible applicator body or substrate 710 can be deformed to follow the contour or curvature of the patient's anatomy. In some embodiments, the alignment notch 720 may be substantially at or near a central location of the flexible substrate 710 and the electrodes 715 are located a fixed distance away from the center notch 720. A tetrapolar electrical measurement is obtained by injecting current between one pair of electrodes 715 and measuring the voltage between the other pair. Alternatively the voltage between one pair of electrodes can be fixed, and the resulting current drawn through the alternate electrodes is measured. The integrated tetrapolar patient interface fixes the relative position of each of the four contacts to prevent variability in operator placement.

The integrated patient interface can be fabricated in multiple sizes from which a single unit can be selected based on the patient's breast characteristics such as inter-nipple distance, chest circumference, cup size, and transverse breast measurement. In an aspect, self adhesive pads (E.1) surround wells (E.2), filled with conductive hydrogel (E.3). These wells provide the electrical interface to the patient. The minimum distance between the conductive hydrogel wells is maintained so that the potential drop across the skin impedance is ignored.

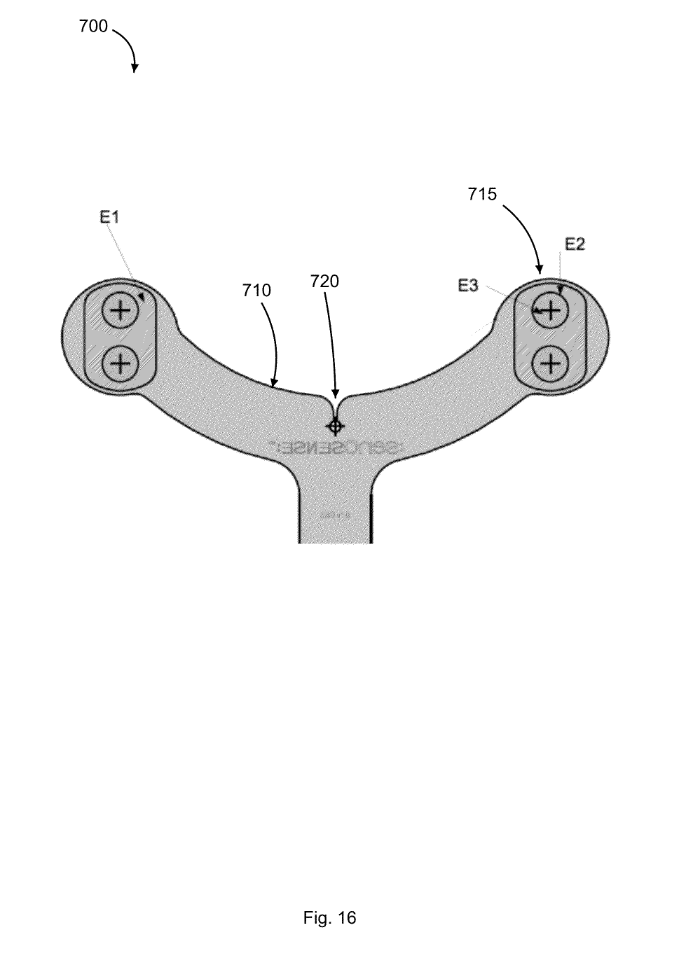

FIG. 16 illustrates a view of an exemplary patient applicator 730, showing its bottom face or patient side (the face proximal to the patient's skin). The conducting traces (E.4) can be generated using an additive process such printable ink such as silver ink or a material removal process such as etching where a conductive layer is eliminated in all but the desired locations. The traces (E.4) are maximally separated to eliminate cross talk and have an adequate cross section to ensure low resistance between the cabal connected and the conductive gel pads described above. The interface between the conductive hydrogel and the traces is made by a material such as Silver-Silver Chloride (E.5) or other material that readily provides ions form electrical conductivity through a gel and biological medium. An electrical connector or interface 740 is provided to electrically couple the patient applicator assembly 730 (and specifically, the electrodes E.5) to other electrical components of the system. A standardized connector format may be employed in some embodiments, which allows for the use of readily available conducting cables with mating end points. The applicator 730 may thus be plugged into and unplugged from the corresponding mating connectors on the conducting cable or ribbon.

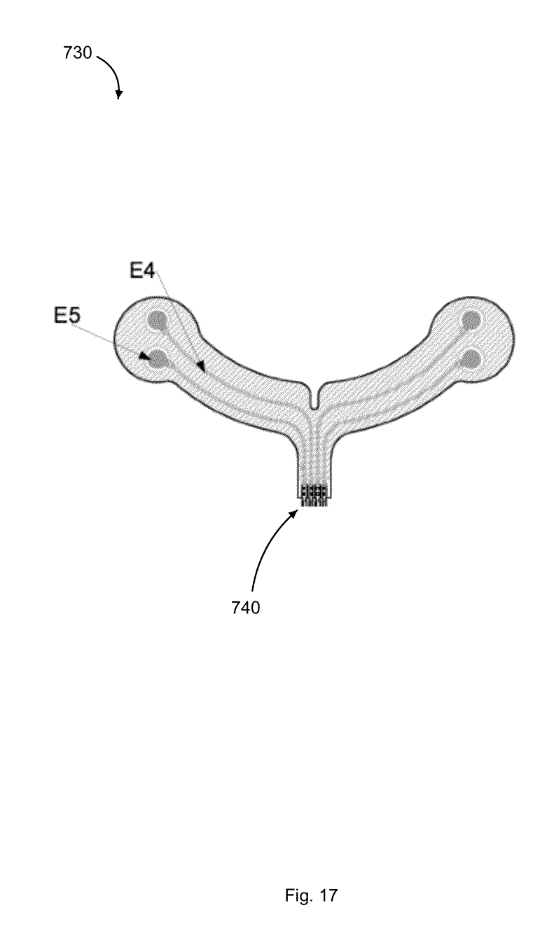

FIG. 17 illustrates the top side (the face away from the patient's skin) of the patient applicator 730. Markings on the top surface of the electrode (E.6) assist the operator in identifying the location of the conductive wells beneath. The conductive wells may be rounded to maximize the contact surface with the breasts. The central alignment notch (E.7) allows the electrode arms (E.8, E.9) to bend and conform to the breast surface (in a plane perpendicular to that of the drawing sheet). The expanded area around the gel wells (E.10) is provided to ensure that the wells are maximally adhered to patient's skin. The electrode is curved so that the sensor can be aligned under the nipple by the contacts can be extended approximately across the center of the breast. In a particular example, a 6″ diameter of curvature is chosen so as not to extend beyond the breast tissue in most (e.g., 95%) of the patient population. As electrical current will extend and disperse throughout the medium, it is not necessary for these contact to circumscribe the complete breast, however larger electrodes can be selected to ensure the complete breast is circumscribed if desired. FIG. 27-101 shows the integrated tetrapolar sensor applied to the breast.

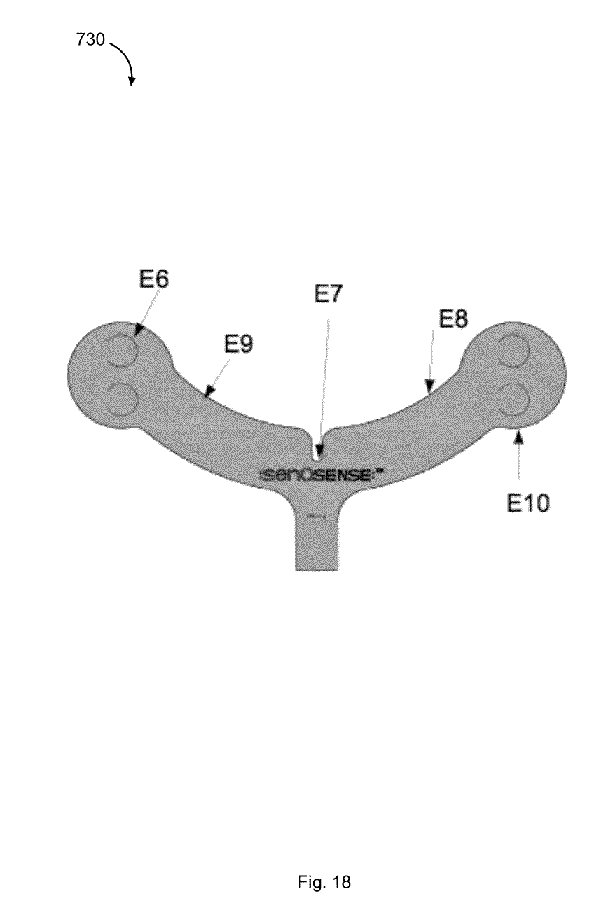

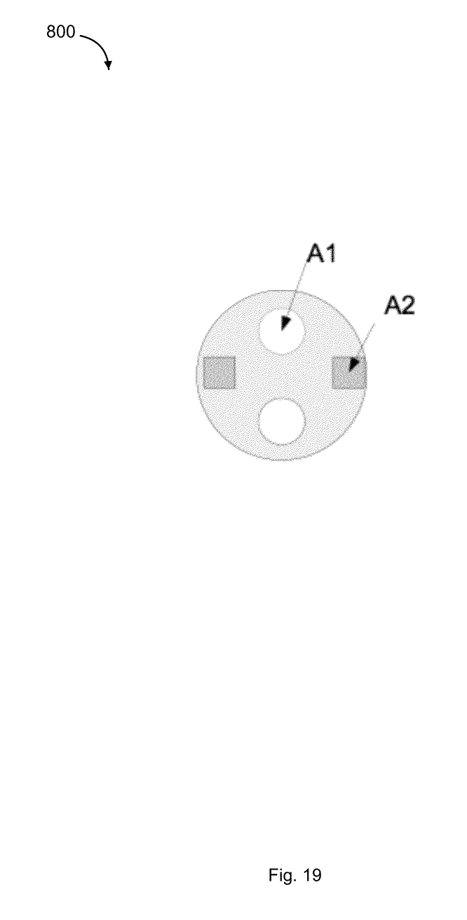

FIG. 18 illustrates an exemplary tetrapolar measurement device 800 in which two integrated two-contact electrodes (A.1) are used and placed on the margins of the breast. The position of the two contacts (A.1) is fixed relative to one another. The connection to the impedance measurement device can be achieved by provided surface contacts (A2) to which an alligator clip can be secured or through a connector (A.4) as illustrated in FIG. 19.

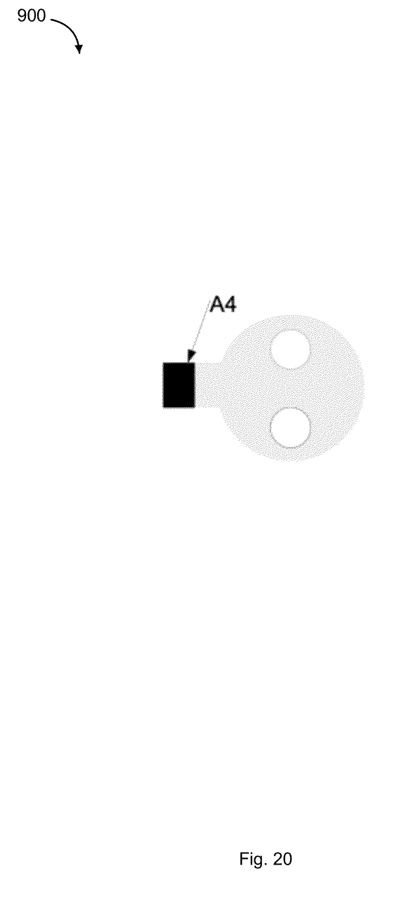

FIG. 20 illustrates another embodiment of a patient applicator template 910 designed to indicate to a practitioner the proper relative positioning of four separate contacts to be placed on a breast using template 910. The template 910 is semi-flexible, flexible, or rigid in construction. It is used to ensure the distance between each electrode is fixed relative to other points on the applicator and to one another. The template 910 includes voids (B1) that are shaped in such a way as to complement the shape of the contact electrodes, with the circular shape of both in this disclosure used for illustration purposes only. In one embodiment, the external template includes a central locating notch (B2) and voids that can used to position the two integrated contact electrodes.

FIG. 21 illustrates another exemplary template applicator 920 in which cutout holes (C1,C2) are placed. Once placed on the patient's body, a marker is used to make marks on the patient's body to assist with electrode placement. The template 920 is not needed after the marks are made and can be discarded or reused. The electrodes can be produced with indicators that can be used to align it to these marks on the patient's body. Therefore, instead of using a permanent patient applicator including the electrical contact points or electrodes described earlier, the template 920 can be used to mark the relative positions for placement of the electrodes, or the electrodes can be positioned in the holes C1, C2 and the template 920 can then be removed.

As mentioned before, connection between the patient applicator and the rest of the patient interface unit can be made by attaching or clamping to an extended tail in the central part of the patient interface unit 30 or one of the edges using standard means for attaching to ribbon cables and flexible PCBs such as ZIF™ connectors or single or dual row headers such as the nicomatic OF series.

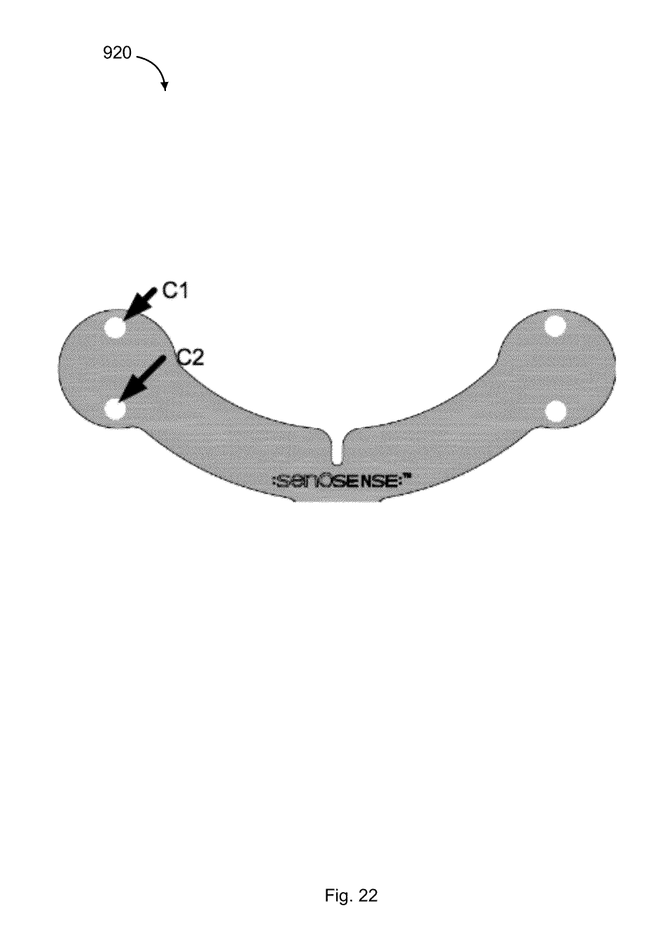

FIG. 22 illustrates steps of a method 990 for computing a future risk of a disease based on non-invasive non-ionizing measurements of properties of an organ or tissue being studied. First, the impedance of the tissue is measured. Next, the impedance is correlated with or converted to a percent (or other) mammographic density value using a mathematical relationship or lookup table as described herein. Then, the mammographic density is used to derive a relative future risk of the disease and determine a relative future risk factor. In addition, other risk factors and results of questionnaires, HRT usage and so on are incorporated. Then the required future risks, for example, 5 year or 10 year risk is computed using the future risk factor in a model as described elsewhere here and in related literature and cited references, which are incorporated by reference. Finally, the patient's risk is multiplied by said relative future risk factor and the future risk factor and mammographic density may be output to an output device or user interface. In some embodiments, the system for performing these steps is substantially contained in one or two physical device modules that can be portable and used by practitioners and/or their patients. In other embodiments other information such as patient breast size, weight, height, transverse breast measurement, nipple spacing etc. are used to further correct the conversion between the measured impedance and breast composition. In further embodiments, applicators with different fixed spacing may be used depending on the patients' characteristics. The conversion from an impedance to a breast composition measurement in these cases would follow a known or estimated relationship, e.g., as shown in FIG. 9 but acquired using the different sized applicator.

It should be noted that the present apparatus and method may be deployed for determining characteristics of tissue other than just the female breast. However, for the sake of illustration, the present is described for measurements made on the breast. Generally, a patient interface unit may include a female patient interface as discussed herein, but other sizes, shapes, and interrogatory signals and algorithms for calculation may be adapted by those skilled in the art to measure properties of other tissues and body parts as well.

Because the present patient applicators are constructed from thin sheets, it is possible to efficiently and economically manufacture these using relatively inexpensive materials and methods. The sheet material can be provided on rolls and the applicator shapes can be cut or stamped from the sheets to maximize the number of applicators that can be produced per unit square area of sheet material. The printing of the electrical contacts on the applicator sheets is likewise done using relatively simple methods.

The device can also calculate hypothetical reductions in risk based interventions including changes in hormonal status (stopping HRT, Stopping OC use, changes in BMI, having a child etc, taking prophylactic drugs such as Tamoxifen). The following tables summarizes interventions that can be used to reduce breast cancer risk. To report a potential risk reduction, the user interface module can take as input the current risk and the potential risk reduction shown in the table below. The reduced risk after a hypothetical intervention is calculated by taking the current risk R1 and multiplying it by 1 minus the percentile risk reduction). For example if a patient's initial risk as calculated above is 25%, to calculate the hypothetical new risk by opting to breast feed, the final risk reduction is 25%*(1−0.23)=19.23%. Similarly if the same patient initiates therapy with Tamoxifen, the risk after the therapy is calculated as 25% (1−0.65)=8.75%.

| | Possible Risk |
|---|---|
| Intervention | Reductions (%) |
| Chemoprevention (Tamoxifen, Raloxifene) | 65 |
| Reduce Alcohol Consumption | 25 |
| Breast Feeding | 23 |
| Physical Activity | 17 |
| Diet modification | 9 |

A system whereby an impedance derived breast composition measurement is used to determine when a patient is benefiting from risk reduction therapy such as that provided by Tamoxifen, Raloxifene or other endocrine therapies as well as other means such as diet, exercise, breast feeding etc. is described. A patient undergoing risk reduction therapy would monitor their breast density at a fixed rate, such as monthly. If there were sources of variation either associated with electrode placement or reader variability there may be too much noise to extract a physiological change from this series of measurements.

When the breast density was reduced substantially, for example by 10%, the risk reduction therapy could be discontinued. Additionally the risk reduction therapy could continue until no further measurable decrease in breast density has been observed for a period (for example one or two consecutive months).

Additionally, if no reduction in breast density occurs after initiating Tamoxifen or other therapy and waiting a sufficient amount of time (3 months), therapy can be discontinued. Alternatively the dosage can be increased from 20 mg to establish whether a higher dose would result in further risk reduction.

Additionally, if no reduction in breast density occurs after initiation Tamoxifen or other therapy, an alternate therapy such as aromatase inhibitor can be initiated.

Additionally, a lower dose of Tamoxifen can be used initially such as 20 mg every second day or 15 mg daily and only increased to 20 mg daily if no change in breast density is observed after a sufficient time (for example 3 months).

In the context of endocrine therapy to prevent a cancer recurrence, Tamoxifen taking patients whose breast density does not decrease are at more than twice the risk of a cancer recurrence in 5 years. Such patients may opt for alternate pharmacological therapies or more aggressive surgical options such as mastectomy.

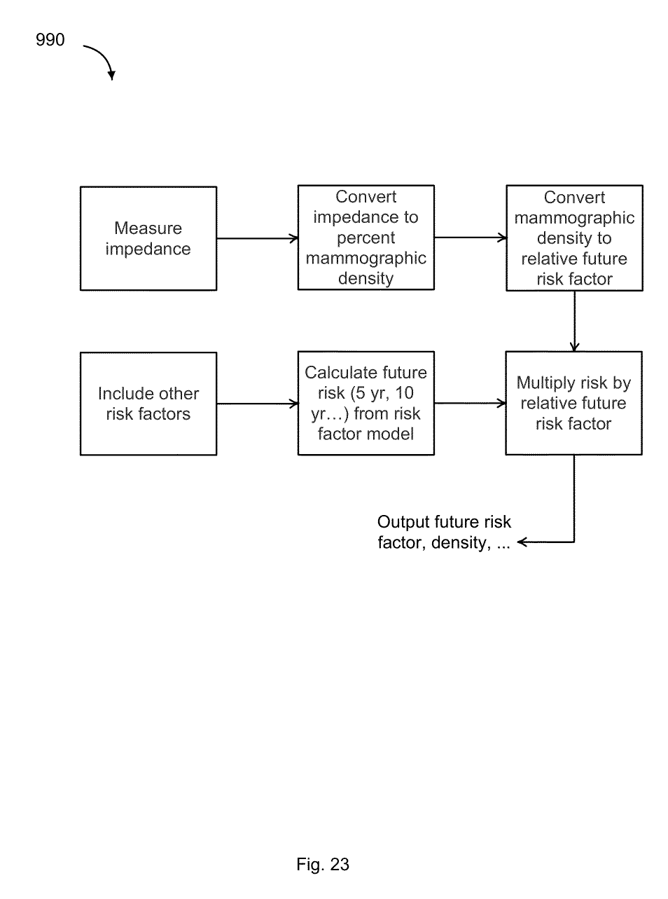

FIG. 23 illustrates exemplary steps of a method 1000 for monitoring the effect of therapy on breast composition and therefore breast cancer risk. Prior to initiating a risk reduction therapy a patient will have an initial breast composition measurement. Following initiation of treatment the patient's breast density will be monitored at a fixed interval defined by the response time of the breast tissue to the treatment, typically 3, 6, or 9 months. After the fixed interval, a second measurement will be made. If the patient's breast composition has not changed favourably, as in an insufficient increase or even decrease in impedance, in that interval, treatment can be modified either through dosage intensification, a new therapy, or abandoned. If breast composition has changed as indicated by an increase impedance, treatment can continue or be completed. In one embodiment of the invention, treatment is continued until there is no statistically significant increase impedance across three successive measurements

An optional database module allows women being monitored for risk intervention as described above to be tracked from visit to visit. A patient's recorded impedance and calculated breast density will be stored with other patient information including information concerning their phase in the menstrual, and other risk information. The reduction in breast density will be displayed, and compared to the expected reduction in breast density at that time period. The relative reduction in breast density can then be reported to the patient in quartiles or according their rank in comparison to other women after the same duration of treatment.

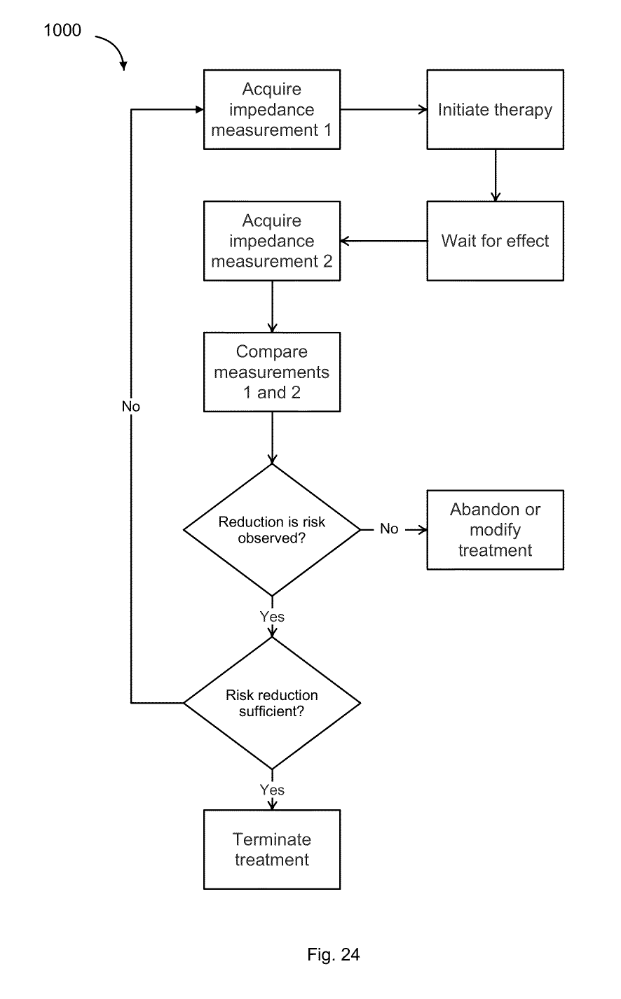

FIG. 24 illustrates another method 1100 in which a women initiating hormone replacement therapy (HRT) such as oestrogen, progesterone or combination therapy delivered orally transdermally in an inhaled mist or otherwise is monitored. HRT has an effect on breast cancer risk and breast density, and that women whose breast density increases have a subsequent increase in breast cancer risk with HRT. The method involves an initial measurement of breast composition using impedance followed by waiting an interval such as 3, 6, or 9 month prior to a second measurement. If breast cancer risk is increased based on a significant increase in breast density, HRT can be discontinued or the type/intensity/route of delivery changed. If breast density has not increased, the women can continue taking HRT and being followed by the process until the next time interval.

Breast density can change throughout the menstrual cycle and based on other factors in the short term. For this reason, the system can incorporate several measurements of density at different points in the menstrual cycle or throughout a week to determine both the baseline and monitored breast density change to ensure that the change is indeed the result of the therapy.

As discussed above, it is useful to measure tissue composition because tissue composition may have predictive power with regard to a person's risk of developing a disease and may help determine whether the person needs additional screening tests. Longitudinal measurement of tissue composition may also enable monitoring of changes in tissue composition that would correspond to changes in disease risk.

In an aspect, the present apparatus and method may be deployed for determining characteristics of tissue other than just the female breast. However, for the sake of illustration, the present is described for measurements made on the breast. Generally, a patient interface unit may include a female patient interface as discussed herein, but other sizes, shapes, and interrogatory signals and algorithms for calculation may be adapted by those skilled in the art to measure properties of other tissues and body parts as well.

The present disclosure is directed to various systems, including an integrated medical apparatus and information device, and methods, which permit obtaining metrics and estimates useful for clinical applications. For example, the present systems and methods permit better monitoring of a risk of a disease. In some embodiments, the disease is breast cancer in women.

Aspects of the systems and methods employ combinations of factors as inputs into a microprocessor-based system coupled to a memory, network, database, interface, or other components. The system can use the present methods, augmented by inputs and data as appreciated by those skilled in the art, to develop an estimate or metric or estimate of a likelihood of a clinically-relevant condition. This can then further drive clinical testing, monitoring, or other intervention and investigation relating to a patient under consideration. Other risk factors are collected and aggregated by the present method in the present system to generate an overall risk score or other useful output that can be communicated to a clinical practitioner or to another health care system or apparatus. Other testing methods, e.g., mammogram results, may also be quantified for use in the present score.

A combination of factors is used, as mentioned, including historical and statistical data, as well as tissue characteristics for the given patient. Breast density is used in some embodiments as such a factor. But generally, any tissue composition at a location of the body of the patient is used as input to the present systems and methods. More specifically, an impedance of tissue can be employed as stated above. The impedance may be electrically derived, or may be derived by a non-electrical measurement. Ultrasound, optics, electromagnetics, or other modalities may be employed to derive said impedance and/or said tissue density information.

The embodiments and description and drawings provided herein are illustrative and allow those skilled in the art to understand the inventions and to incorporate the inventions into systems and methods comprehended by the present disclosure. The present embodiments should therefore not be considered exhaustive or limiting, but other derivative and similar techniques and devices relating hereto should be considered covered by the present scope of invention as well.

Extensions of the above preferred examples are of course available and comprehended by the present inventor. Spatial and geometric configurations beyond those illustrated here would be understood to those skilled in the art upon reviewing the present disclosure. Also, additional configurations and positioning of the electrical elements of the present apparatus can be made still consistent with that described herein. Additionally, other augmented features and optional components and steps can be taken to refine the results obtained with the present system and method.

What is claimed is:

1. A method for breast tissue characterization, comprising:
obtaining a non-invasive electrical measurement of breast tissue composition of a patient; and
estimating a risk of future breast cancer corresponding to at least said non-invasive electrical measurement of said breast tissue composition.

2. The method of claim 1, obtaining said measurement comprising obtaining an electrical impedance.

3. The method of claim 1, obtaining said measurement comprising obtaining an electrical measurement indicative of a relative fat-free mass of said breast tissue.

4. The method of claim 2, obtaining said impedance comprising providing a substantially constant current to said breast tissue using a first pair of conducting electrodes, and detecting a resulting voltage difference in said tissue using a second pair of conducting electrodes, then using the current and voltage to determine said electrical impedance.

5. The method of claim 2, further comprising determining a mammographic breast density of said breast tissue corresponding to said electrical impedance of said breast tissue.

6. The method of claim 5, further comprising computing a future risk of breast cancer from at least said determined mammographic breast density of said breast tissue.

7. The method of claim 6, further comprising factoring other risk factors, beyond said breast density, into the determination of said future risk of breast cancer.

8. The method of claim 1, further comprising associating an effect of a drug delivered to said female patient with said future risk of breast cancer in view of successive such non-invasive electrical measurements.

9. The method of claim 1, practiced on female patients who are not suffering from breast cancer at the time of performing said method and for whom a future risk of breast cancer is nonetheless estimated.

10. The method of claim 1, practiced on a single breast of a female patient.

11. The method of claim 1, further being practiced on both breasts of a female patient and further combining results of said measurements made thereby on each of said breasts into a single estimate of said future risk.

12. The method of claim 1, further comprising comparing said measurement to data in a database to determine how said measurement compares to a population.

13. The method of claim 12, further comprising determining a rank score for said patient relative to said population.

14. The method of claim 1, further comprising comparing a result of said measurement to a pre-determined threshold value and making an assessment regarding said patient's future risk of disease based on said comparison.

15. The method of claim 1, further comprising assigning a property to various values of said measurement and assessing said patient's future risk of disease based on the results of the assigned properties.

16. The method of claim 1, said measurement comprising measurement of electrical impedance, said method further comprising estimating values of mammographic density based on said measurement of electrical impedance and comparing the estimated values of mammographic density to historical data to estimate a future risk of breast cancer therefrom.

17. The method of claim 1, said measurement comprising measurement of electrical impedance, and said method further comprising determining a BiRADS category of said patient corresponding to said measured electrical impedance, and further comprising estimating a future risk of breast cancer therefrom.

18. The method of claim 1, further comprising combining said measurement with a plurality of other patient data corresponding to the future risk, including an age of the patient, and computing an estimate of a future risk of breast cancer for said patient based on said combination.

19. The method of claim 18, further comprising multiplying said future risk of breast cancer by a relative risk corresponding to said patient.

20. A system for determining a risk of future breast cancer in a patient, comprising:
a flexible multi-electrode applicator, having a first surface comprising a plurality of conducting electrodes for making non-invasive non-ionizing electrical contact with a skin tissue of a patient's breast at a corresponding plurality of locations thereon, and having a mechanical index for locating said flexible applicator on said breast with respect to an anatomical feature thereof;
a measuring circuit in electrical communication with said plurality of conducting electrodes that generates an output signal corresponding to an electrical impedance of said breast; and
a processor that receives said output of said measuring circuit and generates a result corresponding to a risk of future breast cancer.

21. The system of claim 20, further comprising a user interface for entering other risk factors for inclusion in said result corresponding to said risk of future breast cancer.

22. The system of claim 20, further comprising a conducting cable adapted for mating to said applicator.

23. An applicator for applying a plurality of conducting electrodes to a portion of a patient's body, comprising:
a thin flexible insulating sheet substrate shaped to conform to said portion of the patient's body;
a plurality of conducting electrode contacts disposed on a first face of said substrate, at least a first pair of which provide a substantially constant current to said portion of the patient's body and at least a second pair of which sense a potential difference (voltage) therebetween;
an electrical connector connecting the conducting electrode contacts to corresponding electrical circuitry providing said current and receiving said voltage; and
a mechanical index for positioning said applicator with respect to said portion of said patient's body and for maintaining said conducting electrodes in relatively fixed relative positions with respect to one another.

24. The applicator of claim 23, generally dimensioned and configured for application to an exterior surface of a female breast and comprising a central positioning mechanical index and a pair of distal arms each attaching two conducting electrodes thereto.

25. The applicator of claim 23, said electrode contacts made to operate without direct contact with a surface of said patient's body.

26. The applicator of claim 23, being from a set of applicators of various sizes, said pairs of electrodes on a given applicator of said set being separated from one another by a substantially fixed spatial separation distance.

27. An applicator template for placement of electrodes on an external surface of a portion of a patient's body, comprising:
an insulating substrate dimensioned and configured to be applied to said portion of said patient's body;
said substrate comprising a plurality of apertures permitting marking of a corresponding plurality of locations on said portion of said patient's body.

28. A method for delivering a treatment to a patient, comprising:
acquiring a first measurement of electrical impedance of a portion of the patient's body;
initiating said treatment on said patient;
waiting a determined length of time for initial results of said treatment;
acquiring a second measurement of electrical impedance of a portion of the portion of the patient's body;
comparing said first and second measurements;
determining if said treatment changes a future risk of disease based on a predetermined criterion including changes in electrical impedance; and
terminating said treatment if the change in risk of future disease in said patient is sufficient, and repeating the above steps if the change of risk is not sufficient.

29. The method of claim 28, further comprising calculating if the risk of future disease based on interventions including changes in hormonal status would be altered.
